(19) United States
(12) Patent Application Publication
(10) Pub. No.: US 2026/0366011 A1
(43) Pub. Date: Nov. 19, 2026

(54) METHODS AND COMPOSITIONS FOR BARCODING CDNA

(71) Applicant: Bio-Rad Laboratories, Inc., Hercules, CA (US)

(72) Inventors: Eric K. POMARANSKI, Plymouth, MI (US); Adam M. McCOY, Davis, CA (US); Jeffrey W. PERRY, Plymouth, MI (US); Niloufar FATTAHI, Ann Arbor, MI (US)

(21) Appl. No.: 19/515,396

(22) PCT Filed: Aug. 30, 2024
(86) PCT No.: PCT/US2024/044859

§ 371 Date: Feb. 27, 2026

Related U.S. Application Data
(60) Provisional application No. 63/535,513, filed on Aug. 30, 2023.

(51) Int. Cl. C12N 15/10; C12Q 1/6806; C12Q 1/686

(52) U.S. Cl. CPC C12N 15/1065; C12N 15/1096; C12Q 1/6806; C12Q 1/686

(57) ABSTRACT

Methods and compositions are provided for preparing RNAs, from a single cell, for sequencing applications. Bead-specific barcoding oligonucleotides are used to introduce a beadspecific barcode sequence, a universal adapter sequence, and optionally one or more tail sequences to cDNA from the single cell. Also provided are plurality of partitions that include a single cell or nucleus or nucleic acids from a single cell or nucleus, beads comprising beadspecific barcoding oligonucleotides, a detection oligonucleotide, a reverse transcriptase, and a DNA polymerase.

### CROSS-REFERENCE TO RELATED APPLICATIONS

This application claims priority to and the benefit of U.S. Provisional Patent Application Ser. No. 63/535,513, filed Aug. 30, 2023, the contents of which are incorporated herein by this reference as if fully set forth herein.

### BACKGROUND OF THE INVENTION

High-throughput RNA sequencing (RNA-seq) is a powerful tool that enables the analysis of the quantity and sequence of RNA in an organism, tissue, or single cell, using next-generation sequencing (NGS) methodologies. Single cell RNA-seq (scRNA-seq) involves the analysis of gene expression in individual cells, which is otherwise lost in bulk RNA studies. Library preparation methods for scRNA-seq, like other RNA-seq methods, typically require reverse transcription of the RNA into cDNA, as well as additional enrichment, purification, and amplification steps (and fragmentation steps, in some methods) to yield a sequencing-ready RNA-seq cDNA library. Each of these steps have the potential to result in a loss of efficiency, and therefore, a loss of data. This loss is particularly critical when analyzing transcripts present at only a few copies per cell. The instant disclosure provides solutions to the potential data loss by, for example, reducing the number of steps required in the library preparation.

### BRIEF SUMMARY OF THE INVENTION

In some embodiments, methods for barcoding cDNAs in a partition are provided. In some embodiments, the method comprises:
providing a plurality of partitions, wherein different partitions comprise at least one bead linked to a plurality of copies of a barcoding oligonucleotide comprising 5′-3′: a first PCR priming sequence, a bead-specific barcode sequence, and a capture sequence; and a single cell, single nucleus, or nucleic acids from a single cell or nucleus;
in the partitions, performing RNA capture, wherein the performing comprises annealing sequences of cellular RNA to some copies of the barcoding oligonucleotides;
performing reverse transcription, wherein performing comprises forming first strand cDNAs by extending the barcoding oligonucleotides with reverse transcriptase using the cellular RNA as a template, thereby creating RNA:DNA duplexes, wherein the first strand cDNAs comprise the first priming sequence, the bead-specific barcode sequence, the capture sequence, and a portion of the target gene sequence;
removing at least a portion of the RNA in the RNA:DNA duplexes;
contacting the first strand cDNAs with a plurality of gene-specific detection oligonucleotides, wherein each gene-specific detection oligonucleotide comprises 5′-3′: a second PCR priming sequence and a reverse complement sequence of a portion of a target gene cDNA reverse complement sequence, wherein the portion is 50-600 nucleotides away from the capture sequence or the DNA equivalent thereof;
performing second strand synthesis to form second strand cDNAs using first strand cDNAs as templates and the gene-specific detection oligonucleotides as primers, thereby forming double-stranded cDNAs of sequencing length from a plurality of different RNAs; and
performing an amplification reaction using a DNA polymerase with the double-stranded cDNAs, a first primer comprising a universal adapter sequence and a bead-specific barcode sequence or reverse complement sequence, and a second primer that comprises a universal adapter sequence and a target gene sequence or reverse complement sequence; thereby producing double stranded sequencing cDNA libraries comprising the universal adapter sequence, the bead-specific barcode sequence, and the portion of the target gene sequence. In certain embodiments, the first primer, the second primer, or both comprise an index sequence, thereby forming double stranded sequencing cDNAs comprising the index sequence.

In some embodiments, the capture sequence is a poly T sequence. In certain embodiments, the capture sequence comprises 22-36 Ts. In other embodiments, the capture sequence is a portion of a target gene sequence.

In some embodiments, the reverse transcription is performed in the partition. In some embodiments, both reverse transcription and second strand synthesis are performed in the partition.

In some embodiments, the plurality of gene-specific detection oligonucleotides comprises at least 100 gene-specific detection oligonucleotides specific for different mRNAs present in the single cell. In some embodiments, at least one gene-specific detection oligonucleotide of the plurality of gene-specific detection oligonucleotides is specific for a target gene that is known to be expressed in the single cell.

In some embodiments, at least one gene-specific detection oligonucleotide binds to a sequence that is 100 to 400 nucleotides upstream of the mRNA poly A tail. In certain embodiments, at least one gene-specific detection oligonucleotide binds to a sequence that is 180 to 220 nucleotides upstream of the mRNA poly A tail.

In some embodiments, the providing a plurality of partitions comprises providing partitions comprising intact cells and subsequently lysing the cells in the partitions.

In some embodiments, the reverse transcription and the RNA:DNA duplex degradation are performed by a single enzyme having RNAse H′ activity.

In some embodiments, after performing second strand synthesis, the methods comprise inactivating the polymerase and reverse transcriptase in or out of the partitions. In some embodiments, the inactivating comprises applying heat to the partitions or to a bulk mixture. In certain embodiments, the inactivating comprises incubating the partitions or a bulk mixture at 75-90 degrees Celsius.

In some embodiments, the partitions are droplets in an emulsion or microwells.

In some embodiments, the cell is a mammalian cell.

In some embodiments, the bead is a hydrogel bead.

In some embodiments, the disclosure provides a plurality of partitions. In some embodiments, the different partitions comprise at least one bead linked to a plurality of copies of a barcoding oligonucleotide comprising 5′-3′: a shared priming sequence, a bead-specific barcode sequence, and a capture sequence; and a single cell, single nucleus, or nucleic acids from a single cell or nucleus. In other embodiments, the partitions further include a plurality of gene-specific detection oligonucleotides, wherein each gene-specific detection oligonucleotide comprises 5′-3′: a shared priming reverse complement sequence and a portion of a target gene reverse complement sequence wherein the portion is 50-600 nucleotides away from the capture sequence or the DNA equivalent thereof; a reverse transcriptase; and optionally a polymerase. In some embodiments, the partitions are droplets in an emulsion or microwells. In some embodiments, the cell is a mammalian cell. In some embodiments, the bead is a hydrogel bead.

In some embodiments, methods of barcoding cDNAs from cells in a partition are provided. In some embodiments, the method comprises:
providing a plurality of partitions, wherein different partitions comprise:
   (i) at least one bead linked to a plurality of copies of a first barcoding oligonucleotide and a plurality of copies of a second barcoding oligonucleotide; wherein the first barcoding oligonucleotide comprises:
   a first PCR priming sequence, a bead-specific barcode sequence, a capture sequence, and a site labile to a single stranded break or nick;
   and wherein the second barcoding oligonucleotide comprises 5′-3′: a second PCR priming sequence, a bead-specific barcode sequence, and a tail capture sequence;
   (ii) a plurality of cleavable-tailed gene-specific detection oligonucleotides, wherein each gene-specific detection oligonucleotide comprises: a cleavable tail sequence and a portion of a target gene DNA reverse complement sequence that binds to the cDNA 50-600 bp away from the capture sequence;
   (iii) a reverse transcriptase; and
   (iv) a single cell, nucleus, or nucleic acids from a single cell or nucleus;
in the partitions, performing reverse transcription, wherein the performing comprises annealing sequences of the cellular RNAs to some copies of the first barcoding oligonucleotides and forming first strand cDNAs by extending the first barcoding oligonucleotides with the reverse transcriptase using the cellular RNAs as a template, thereby creating RNA:DNA duplexes, wherein the first strand cDNAs comprise the first PCR priming sequence, the bead-specific barcode sequence, the capture sequence, the site labile to a single stranded break or nick, and the target gene sequence;
in the partitions, degrading the RNA in the RNA:DNA duplexes;
in the partitions, performing a second strand synthesis to form second strand gene-specific cDNAs including the cleavable tail using first strand cDNAs as templates and the gene-specific detection oligonucleotides as primers, thereby forming double-stranded cDNAs wherein the tailed second strand gene-specific cDNAs comprise: the cleavable tail sequence, the target gene reverse complement sequence, the bead-specific barcode reverse complement sequence, and the first PCR priming reverse complement sequence;
in the partitions, producing a single stranded break at the labile site, thereby creating a free 3′ hydroxyl group at the site;
in the partitions, performing a strand synthesis with a displacing polymerase to form DNA-tailed first strand cDNAs using the second strand gene-specific cDNAs as templates and the free 3′ hydroxyl group to begin extension, thereby forming tailed double-stranded cDNAs, wherein the DNA-tailed first strand cDNAs comprise: the first PCR priming sequence, the bead-specific barcode sequence, the target gene sequence, and a DNA tail sequence that is reverse complementary to the cleavable tail sequence;
in the partitions, cleaving the cleavable tail sequence in the double stranded cDNAs, thereby producing a double-stranded cDNA with a single-stranded DNA tail on the 3′ end of the first strand;
in the partitions, annealing the DNA tail of the double-stranded cDNA to the second barcoding oligonucleotide and performing a strand synthesis using the second barcoding oligonucleotide as templates and the single-stranded DNA tail of the first strand cDNA to begin extension;
in the partitions, performing a strand synthesis with a displacing polymerase to form double-stranded cDNAs comprising: the first PCR priming sequence, the bead-specific barcode sequence, the truncated capture sequence, the target gene sequence, the DNA tail sequence, the bead-specific barcode reverse complement sequence, and the second PCR priming reverse complement sequence;
generating a bulk mixture by combining contents of the partitions; and performing a PCR reaction with primers that bind to the first or second PCR priming sequence or reverse complement sequence, thereby forming double stranded sequencing cDNA libraries comprising: the PCR priming sequence, the bead-specific barcode sequence, the capture sequence, the target gene sequence, the DNA tail sequence, the bead-specific barcode reverse complement sequence, and the PCR priming reverse complement sequence.

In some embodiments of these methods, after performing the PCR reaction, the methods further comprise determining the nucleotide sequence of the double-stranded sequencing cDNAs, wherein if two different bead-specific barcode sequences are present on the same cDNA, then sequencing reads comprising either of the two bead-specific barcode sequences are from the same partition.

In some embodiments, the cleavable tail sequence comprises a plurality of RNA nucleotides. In some embodiments, the cleavable tail is cleaved by RNase H⁺ activity in the partitions. In some embodiments, the capture sequence is a poly T sequence. In other embodiments, the capture sequence includes a portion of a target gene sequence.

In some embodiments, the site labile to a single stranded break or nick comprises at least one RNA nucleotide. In some embodiments, RNase H⁺ activity produces the single stranded break at the site. In certain embodiments, the site labile to a single stranded break or nick comprises at least one uracil nucleotide in a poly T sequence.

In some embodiments, the plurality of gene-specific detection oligonucleotides comprises at least 100 gene-specific detection oligonucleotides specific for different mRNAs present in the single cell. In some embodiments, at least one gene-specific detection oligonucleotide of the plurality of gene-specific detection oligonucleotides is specific for a target gene that is known to be expressed in the single cell or nucleus.

In some embodiments, at least one gene-specific detection oligonucleotide binds to a sequence that is 100 to 400 nucleotides upstream of the mRNA poly A tail. In certain embodiments, at least one gene-specific detection oligonucleotide binds to a sequence that is 180 to 220 nucleotides base pairs upstream of the mRNA poly A tail.

In some embodiments, the providing a plurality of partitions comprises providing partitions comprising intact cells and subsequently lysing the cells in the partitions.

In some embodiments, the reverse transcription, the RNA:DNA duplex degradation, and second strand synthesis are performed by a single enzyme having RNase H⁺ activity. In other embodiments, the reverse transcription and the RNA:DNA duplex degradation are performed by a first enzyme having RNase H′ activity, and the second strand synthesis and DNA-dependent DNA synthesis are performed by a second enzyme.

In some embodiments, after performing the last strand synthesis the methods further comprise inactivating the polymerase and reverse transcriptase in the partitions. In some embodiments, the inactivating comprises applying heat to the partitions. In certain embodiments, the inactivating comprises incubating the partitions at 75-90 degrees Celsius.

In some embodiments, the primers used in the PCR reaction include an index sequence, thereby forming double stranded sequencing cDNAs comprising the index sequence.

In some embodiments, the partitions are droplets in an emulsion or microwells.

In some embodiments, the cell is a mammalian cell.

In some embodiments, the bead is a hydrogel bead.

In some embodiments, the disclosure provides a plurality of partitions. In some embodiments, the different partitions comprise at least one bead linked to a plurality of copies of a first barcoding oligonucleotide and a plurality of copies of a second barcoding oligonucleotide; wherein the first barcoding oligonucleotide comprises: a first PCR priming sequence, a bead-specific barcode sequence, a capture sequence, and a site labile to a single stranded break or nick; and wherein the second barcoding oligonucleotide comprises 5′-3′: a second PCR priming sequence, a bead-specific barcode sequence, and a tail capture sequence; a plurality of cleavable-tailed gene-specific detection oligonucleotides, wherein each gene-specific detection oligonucleotide comprises: a cleavable tail sequence and a portion of a target gene DNA reverse complement sequence that binds to the cDNA 50-600 bp away from the capture sequence; a reverse transcriptase; and a single cell, single nucleus, or nucleic acids from a single cell or nucleus. In some embodiments, the partitions are droplets in an emulsion or microwells. In some embodiments, the cell is a mammalian cell. In some embodiments, the bead is a hydrogel bead.

In some embodiments, methods of barcoding cDNAs from cells in a partition are provided. In some embodiments, the method comprises:
providing a plurality of partitions, wherein different partitions comprise:
   (i) at least one bead linked to a plurality of copies of a first barcoding oligonucleotide and a plurality of copies of a second barcoding oligonucleotide, wherein the first barcoding oligonucleotide comprises 5′-3′: a first PCR priming sequence, a bead-specific barcode sequence, and a first tail capture sequence; and wherein the second barcoding oligonucleotide comprises 5′-3′: a second PCR priming sequence, the bead-specific barcode sequence, and a second tail capture sequence;
   (ii) a plurality of pairs of cleavable-tailed gene-specific detection oligonucleotides, wherein each pair comprises a first gene-specific detection oligonucleotide that comprises a first cleavable tail sequence and a target gene sequence and a second gene-specific detection oligonucleotide that comprises a second cleavable tail sequence and a target gene capture sequence, wherein the first and second gene-specific detection oligonucleotides bind to different sequences of the target gene, and wherein the first gene-specific detection oligonucleotide binds to a sequence that is 50 to 600 nucleotides from the sequence to which the second gene-specific detection oligonucleotide binds;
   (iii) a reverse transcriptase; and
   (iv) a single cell, single nucleus, or nucleic acids from a single cell or nucleus;
in the partitions, performing a reverse transcription, wherein the performing comprises annealing the first gene-specific detection oligonucleotide to cellular RNAs from the cell or nucleus and forming first strand cDNAs by extending the gene-specific detection oligonucleotides with the reverse transcriptase using the cellular RNAs as a template, thereby creating RNA:DNA duplexes, wherein the first strand cDNAs comprise the first cleavable tail sequence and the target gene sequence;
in the partitions, degrading the RNA in the RNA:DNA duplexes;
in the partitions, performing a second strand synthesis to form second strand CDNAs using first strand cDNAs as templates and the second gene-specific detection oligonucleotides as primers, thereby forming double-stranded cDNAs, wherein the second strand cDNAs comprise the second cleavable tail sequence, the target gene capture sequence, and a first DNA tail sequence that is a reverse complement sequence to the first cleavable tail sequence;
in the partitions, cleaving the first cleavable tail, thereby creating a single-stranded first DNA tail sequence on the second strand cDNAs;
in the partitions, annealing the tail capture sequence of the first barcoding oligonucleotide to the single-stranded first DNA tail sequence on the second strand cDNAs and performing strand synthesis using a displacing polymerase to form double tailed first strand cDNAs using the second strand cDNAs as templates and the first barcoding oligonucleotide to begin extension, wherein the double tailed first strand cDNAs comprise the first PCR priming sequence, the bead-specific barcode sequence, the first tail capture sequence, the target gene sequence, and the second DNA tail sequence that is a reverse complement sequence to the second cleavable tail sequence; and performing strand synthesis using the first barcoding oligonucleotide as a template and the first DNA tail sequence to begin extension;
in the partitions, degrading the second cleavable tail sequence, thereby creating a single-stranded second DNA tail sequence;
in the partitions, annealing the tail capture sequence of the second barcoding oligonucleotide to the single-stranded second DNA tail sequence and performing strand synthesis using a displacing polymerase and the double tailed first strand cDNAs as templates and the second barcoding oligonucleotide to begin extension, thereby forming double tailed second strand cDNAs comprising the PCR priming reverse complement sequence, the bead-specific barcode sequence, the second tail capture sequence, the target gene reverse complement sequence, the first DNA tail sequence, a bead-specific barcode reverse complement sequence, and the second PCR priming reverse complement sequence; and performing strand synthesis to form a double tailed first strand cDNA using the second barcoding oligonucleotide as a template and the second DNA tail sequence to begin extension;
generating a bulk mixture by combining contents of the partitions; and performing a PCR reaction with primers that bind to the PCR priming sequences or reverse complement sequences, thereby forming double stranded sequencing cDNA libraries, wherein the cDNAs comprise the first PCR priming sequence, the bead-specific barcode sequence, the first tail capture sequence, the target gene sequence, the second tail sequence, the bead-specific barcode reverse complement sequence, and the second PCR priming sequence.

In some embodiments, the partitions further comprise a polymerase.

In some embodiments, the first cleavable tail sequence, the second cleavable tail sequence, or both comprise a plurality of RNA nucleotides.

In some embodiments, after performing the PCR reaction, the methods further comprise determining the nucleotide sequence of the cDNA libraries, wherein if two different bead-specific barcode sequences are present on the same cDNA, then sequencing reads comprising either of the two bead-specific barcode sequences are from the same partition.

In some embodiments, the plurality of gene-specific detection oligonucleotides comprises at least 100 gene-specific detection oligonucleotides specific for different mRNAs present in the single cell. In some embodiments, at least one gene-specific detection oligonucleotide of the plurality of gene-specific detection oligonucleotides is specific for a target gene that is known to be expressed in the single cell.

In some embodiments, the first gene-specific detection oligonucleotide binds to a sequence that is 100 to 400 nucleotides upstream of the sequence to which the second gene-specific detection oligonucleotide binds. In certain embodiments, the first gene-specific detection oligonucleotide binds to a sequence that is 180 to 220 nucleotides upstream of the sequence to which the second gene-specific detection oligonucleotide binds.

In some embodiments, the providing a plurality of partitions comprises providing partitions comprising intact cells and subsequently lysing the cells in the partitions.

In some embodiments, the reverse transcription, the RNA:DNA duplex degradation, and second strand synthesis are performed by a single enzyme having RNase H⁺ activity. In other embodiments, the reverse transcription and the RNA:DNA duplex degradation are performed by a first enzyme having RNase H^{|} activity, and wherein the second strand synthesis and DNA-dependent DNA synthesis are performed by a second enzyme.

In some embodiments, after performing the last strand synthesis, the methods further comprise inactivating the polymerase and reverse transcriptase in the partitions. In some embodiments, the inactivating comprises applying heat to the partitions. In certain embodiments, the inactivating comprises incubating the partitions at 75-90 degrees Celsius.

In some embodiments, the primers used in the PCR reaction comprise an index sequence, thereby forming double stranded sequencing cDNAs comprising the index sequence.

In some embodiments, the partitions are droplets in an emulsion or microwells.

In some embodiments, the cell is a mammalian cell.

In some embodiments, the bead is a hydrogel bead.

In some embodiments, the disclosure provides a plurality of partitions. In some embodiments, the different partitions comprise at least one bead linked to a plurality of copies of a first barcoding oligonucleotide and a plurality of copies of a second barcoding oligonucleotide, wherein the first barcoding oligonucleotide comprises 5′-3′: a first PCR priming sequence, a bead-specific barcode sequence, and a first tail capture sequence; and wherein the second barcoding oligonucleotide comprises 5′-3′: a second PCR priming sequence, the bead-specific barcode sequence, and a second tail capture sequence; a plurality of pairs of cleavable-tailed gene-specific detection oligonucleotides, wherein each pair comprises a first gene-specific detection oligonucleotide that comprises a first cleavable tail sequence and a target gene sequence and a second gene-specific detection oligonucleotide that comprises a second cleavable tail sequence and a target gene capture sequence, wherein the first and second gene-specific detection oligonucleotides bind to different sequences of the target gene, and wherein the first gene-specific detection oligonucleotide binds to a sequence that is 50 to 600 nucleotides from the sequence to which the second gene-specific detection oligonucleotide binds; a reverse transcriptase; and a single cell, single nucleus, or nucleic acids from a single cell or nucleus. In some embodiments, the partitions are droplets in an emulsion or microwells. In some embodiments, the cell is a mammalian cell. In some embodiments, the bead is a hydrogel bead.

In some embodiments, methods of barcoding cDNAs from cells in a partition are provided. In some embodiments, the method comprises:
providing a plurality of partitions, wherein different partitions comprise:
   (i) at least one bead linked to a plurality of copies of a barcoding oligonucleotide, wherein the barcoding oligonucleotide comprises 5′-3′: a PCR priming sequence, a bead-specific barcode sequence, and a tail capture sequence;
   (ii) a plurality of pairs of gene-specific detection oligonucleotides, wherein each pair comprises a first gene-specific detection oligonucleotide that comprises a cleavable tail sequence and a target gene sequence, and a second gene-specific detection oligonucleotide that comprises a second PCR priming reverse complement sequence and a target gene reverse complement sequence, wherein the first and second gene-specific detection oligonucleotides bind to different sequences of the target gene, and wherein the first gene-specific detection oligonucleotide binds to a sequence that is 50 to 600 nucleotides from the sequence to which the second gene-specific detection oligonucleotide binds;
   (iii) a reverse transcriptase; and
   (iv) a single cell, single nucleus, or nucleic acids from a single cell or nucleus;
in the partitions, performing a reverse transcription, wherein the performing comprises annealing the first gene-specific detection oligonucleotide to cellular RNAs from the cell or nucleus and forming cleavable tailed first strand gene-specific cDNAs by extending the first gene-specific detection oligonucleotides with reverse transcriptase using the cellular RNAs as a template, thereby creating RNA:DNA duplexes, wherein the cleavable tailed first strand gene-specific cDNAs comprise the cleavable tail sequence and the target gene sequence;
in the partitions, degrading the RNA in the RNA:DNA duplexes;
in the partitions, performing second strand synthesis to form second strand cDNAs using cleavable tailed first strand cDNAs as templates and the second gene-specific detection oligonucleotides as primers, thereby forming double-stranded cDNAs, wherein the second strand cDNAs comprise the second PCR priming reverse complement sequence, the target gene reverse complement sequence, and a DNA tail sequence that is a reverse complement sequence to the cleavable tail sequence;
in the partitions, cleaving the cleavable tail, thereby creating a single-stranded DNA tail sequence on the second strand cDNAs;
in the partitions, annealing the tail capture sequence of the barcoding oligonucleotide to the single-stranded DNA tail sequence on the second strand cDNAs and performing strand synthesis using a displacing polymerase to form tailed first strand cDNAs using the second strand cDNAs as templates and the barcoding oligonucleotide as a primer to begin extension, wherein the tailed first strand cDNAs comprise the first PCR priming sequence, the bead-specific barcode sequence, the tail capture sequence, the target gene sequence, and the second PCR priming sequence; and performing strand synthesis to form a tailed second strand cDNA using the barcoding oligonucleotide as a template and the DNA tail sequence as a primer to begin extension;
generating a bulk mixture by combining contents of the partitions; and
performing a PCR reaction with primers that bind to the first or second PCR priming sequences or reverse complement sequences, thereby forming double stranded sequencing cDNA libraries, wherein the cDNAs comprise the first PCR priming sequence, the bead-specific barcode sequence, the tail capture sequence, the target gene sequence, and the second PCR priming sequence.

In some embodiments, the partitions further comprise a polymerase.

In some embodiments, the cleavable tail sequence comprises a plurality of RNA nucleotides. In some embodiments, the cleavable tail sequence is cleaved by RNase H′ activity in the partitions.

In some embodiments, the plurality of gene-specific detection oligonucleotides comprises at least 100 gene-specific detection oligonucleotides specific for different mRNAs present in the single cell or nucleus. In some embodiments, at least one gene-specific detection oligonucleotide of the plurality of gene-specific detection oligonucleotides is specific for a target gene that is known to be expressed in the single cell or nucleus.

In some embodiments, the first gene-specific detection oligonucleotide binds to a sequence that is 100 to 400 nucleotides upstream of the sequence to which the second gene-specific detection oligonucleotide binds. In certain embodiments, the first gene-specific detection oligonucleotide binds to a sequence that is 180 to 220 nucleotides upstream of the sequence to which the second gene-specific detection oligonucleotide binds.

In some embodiments, the providing a plurality of partitions comprises providing partitions comprising intact cells and subsequently lysing the cells in the partitions.

In some embodiments, the reverse transcription, the RNA:DNA duplex degradation, and second strand synthesis are performed by a single enzyme having RNase H⁺ activity. In other embodiments, the reverse transcription and the RNA:DNA duplex degradation are performed by a first enzyme having RNase H⁺ activity, and wherein the second strand synthesis and DNA-dependent DNA synthesis are performed by a second enzyme.

In some embodiments, after performing the last strand synthesis, the methods further comprise inactivating the polymerase and reverse transcriptase in the partitions. In some embodiments, the inactivating comprises applying heat to the partitions. In some embodiments, the inactivating comprises incubating the partitions at 75-90 degrees Celsius.

In some embodiments, the primers used in the PCR reaction comprise an index sequence, thereby forming double stranded sequencing cDNAs comprising the index sequence.

In some embodiments, the partitions are droplets in an emulsion or microwells.

In some embodiments, the cell is a mammalian cell.

In some embodiments, the bead is a hydrogel bead.

In some embodiments, the disclosure provides a plurality of partitions. In some embodiments, the different partitions comprise at least one bead linked to a plurality of copies of a barcoding oligonucleotide, wherein the barcoding oligonucleotide comprises 5′-3′: a PCR priming sequence, a bead-specific barcode sequence, and a tail capture sequence; a plurality of pairs of gene-specific detection oligonucleotides, wherein each pair comprises a first gene-specific detection oligonucleotide that comprises a cleavable tail sequence and a target gene sequence, and a second gene-specific detection oligonucleotide that comprises a second PCR priming reverse complement sequence and a target gene reverse complement sequence, wherein the first and second gene-specific detection oligonucleotides bind to different sequences of the target gene, and wherein the first gene-specific detection oligonucleotide binds to a sequence that is 50 to 600 nucleotides from the sequence to which the second gene-specific detection oligonucleotide binds; a reverse transcriptase; and a single cell, single nucleus, or nucleic acids from a single cell or nucleus. In some embodiments, the partitions are droplets in an emulsion or microwells. In some embodiments, the cell is a mammalian cell. In some embodiments, the bead is a hydrogel bead.

### BRIEF DESCRIPTION OF THE DRAWINGS

FIGS. 1A-1C are schematic drawings of compositions and method steps for one embodiment of the disclosed scRNA-seq library preparation methods. FIG. 1A depicts a bead-specific barcoding oligonucleotide and a gene-specific detection oligonucleotide. FIGS. 1B-1C depict steps involved in the generation of the sequencing library in which each double-stranded cDNA is tagged with the bead-specific barcode sequence. The method steps in FIGS. 1A-1C are shown using GADPH as an example mRNA. FIGS. 1D-IF show the effectiveness of one embodiment of the library preparation methods using IgM transcript as an example. FIGS. 1D and 1E provide the results of High Sensitivity D1000 screen tape analysis (Agilent) showing the amount of target transcripts produced by PCR only (FIG. 1D) or produced by one embodiment of the library preparation methods (FIG. 1E). FIG. 1F is a graph showing the strength of the method for assembling the target region of the transcripts (productive IgM assemblies (left panel) or complete CDR3 regions (right panel)). The results for the PCR only method are shown as the left bar in both panels of FIG. 1F, and the results of two replicates of the embodiment of the library preparation methods are shown in the two right bars in both panels.

productive IgM assemblies were recovered from less than 10 of them in the PCR only Approach 2 (FIG. 1F, left panel), while approximately 100 were recovered using the disclosed Approach 1. This trend was also prevalent when analyzing for complete CDR3 regions (FIG. 1F, right panel)

FIGS. 2A-2E are schematic drawings of compositions and method steps for another embodiment of the disclosed scRNA-seq library preparation methods. FIG. 2A depicts a bead-specific barcoding oligonucleotide for mRNA capture, a bead-specific barcoding oligonucleotide for capturing a tail sequence, and a gene-specific detection oligonucleotide comprising a cleavable tail sequence. FIGS. 2B-2E depict steps involved in the generation of the sequencing library in which each double-stranded cDNA is tagged with the bead-specific barcode sequence and the tail sequence. The method steps are shown using GADPH as an example mRNA.

FIGS. 3A-3E are schematic drawings of compositions and method steps for another embodiment of the disclosed scRNA-seq library preparation methods. FIG. 3A depicts a first bead-specific barcoding oligonucleotide with a first tail capture sequence, a second bead-specific barcoding oligonucleotide with a second tail capture sequence, a gene-specific detection oligonucleotide with a gene-specific sequence and a first cleavable tail sequence, and a gene-specific detection oligonucleotide with a target gene capture sequence and a second cleavable tail sequence. FIGS. 3B-3E depict steps involved in the generation of the sequencing library in which each double-stranded cDNA is tagged with the bead-specific barcode sequence and both tail sequences. The method steps are shown using GADPH as an example mRNA.

FIGS. 4A-4D are schematic drawings of compositions and method steps for another embodiment of scRNA-seq library preparation. FIG. 4A depicts a bead-specific barcoding oligonucleotide with a first tail capture sequence, a first gene-specific detection oligonucleotide with a gene-specific capture sequence and a cleavable tail, and a second gene-specific detection oligonucleotide with a gene-specific reverse complement sequence and a universal adapter reverse complement sequence. FIGS. 4B-4D depict steps involved in the generation of the sequencing library in which each double-stranded cDNA is tagged with the bead-specific barcode sequence and the tail sequence. The method steps are shown using GADPH as an example mRNA.

### DEFINITIONS

Unless defined otherwise, all technical and scientific terms used herein generally have the same meaning as commonly understood by one of ordinary skill in the art to which this disclosure belongs. Generally, the nomenclature used herein and the laboratory procedures in cell culture, molecular genetics, organic chemistry, and nucleic acid chemistry and hybridization described below are those well-known and commonly employed in the art. Standard techniques are used for nucleic acid synthesis. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a bead” includes a plurality of such beads and reference to “the sequence” includes reference to one or more sequences known to those skilled in the art, and so forth.

An “oligonucleotide” is a polynucleotide. Generally, oligonucleotides will have fewer than 250 nucleotides, in some embodiments, between 4-200, e.g., 10-150 nucleotides.

A “primer” refers to a polynucleotide sequence that hybridizes to a sequence on a target nucleic acid and serves as a point of initiation of nucleic acid synthesis. Primers can be of a variety of lengths and are often less than 50 nucleotides in length, for example 12-30 nucleotides, in length. The length and sequences of primers for use in PCR can be designed based on principles known to those of skill in the art, see, e.g., PCR Protocols: A Guide to Methods and Applications (Innis et al., eds., 1990). Primers can be DNA, RNA, or a chimera of DNA and RNA portions. In some cases, primers can include one or more modified or non-natural nucleotide bases. In some cases, primers are labeled.

“Primer extension” refers to any method in which a primer is extended in a template-specific manner. Examples of primer extension include, for example, methods in which a primer hybridizes to a template nucleic acid and a polymerase extends the primer in a template-specific manner. In some embodiments, the method is referred to as “strand synthesis.” In some embodiments, the template is DNA, and the polymerase is a DNA polymerase. In some embodiments, the template is RNA, and the polymerase is a reverse-transcriptase. As used herein, “reverse transcription” is a method that copies RNA into DNA. Primer extension can also include, for example, template switching (see, e.g., Zhu Y Y, Machleder E M, et al. (2001) Biotechniques, 30 (4): 892-897; Ramskold D, Luo S, et al. (2012) Nat Biotechnol, 30 (8): 777-78, and nick polymerization (also referred to as nick translation), the latter involving nicking one strand of a nucleic acid duplex and using the nicked strand as a primer that is extended using the other strand as a template (see, e.g., Leonard G. Davis Ph.D., et al, in Basic Methods in Molecular Biology, 1986).

A nucleic acid, or a portion thereof, “hybridizes” or “anneals” to another nucleic acid under conditions such that non-specific hybridization is minimal at a defined temperature in a physiological buffer (e.g., pH 6-9, 25-150 mM chloride salt). In some cases, a nucleic acid, or portion thereof, hybridizes to a conserved sequence shared among a group of target nucleic acids. In some cases, a primer, or portion thereof, can hybridize to a primer binding site if there are at least about 6, 8, 10, 12, 14, 16, or 18 contiguous complementary nucleotides, including “universal” nucleotides that are complementary to more than one nucleotide partner. Alternatively, a primer, or portion thereof, can hybridize to a primer binding site if there are fewer than 1 or 2 complementarity mismatches over at least about 12, 14, 16, or 18 contiguous complementary nucleotides. In some embodiments, the defined temperature at which specific hybridization occurs is room temperature. In some embodiments, the defined temperature at which specific hybridization occurs is higher than room temperature. In some embodiments, the defined temperature at which specific hybridization occurs is at least about 37, 40, 42, 45, 50, 55, 60, 65, 70, 75, or 80 °C. In some embodiments, the defined temperature at which specific hybridization occurs is 37, 40, 42, 45, 50, 55, 60, 65, 70, 75, or 80 °C.

A “template” refers to a polynucleotide sequence that comprises the polynucleotide to be copied or amplified, flanked by or a pair of primer hybridization sites. Thus, a “target template” comprises the target polynucleotide sequence adjacent to at least one hybridization site for a primer. In some cases, a “target template” comprises the target polynucleotide sequence flanked by a hybridization site for a “forward” primer and a “reverse” primer. In some embodiments, a “PCR priming sequence” refers to a site to which a forward primer or reverse primer binds for PCR amplification.

As used herein, “nucleic acid” means DNA, RNA, single-stranded, double-stranded, or more highly aggregated hybridization motifs, and any chemical modifications thereof. Modifications include, but are not limited to, those providing chemical groups that incorporate additional charge, polarizability, hydrogen bonding, electrostatic interaction, points of attachment and functionality to the nucleic acid ligand bases or to the nucleic acid ligand as a whole. Such modifications include, but are not limited to, peptide nucleic acids (PNAs), phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Nucleic acids can also include non-natural bases, such as, for example, nitroindole. Modifications can also include 3′ and 5′ modifications including but not limited to capping with a fluorophore (e.g., quantum dot) or another moiety. As used herein, the terms “reverse complement” or “reverse complementary sequence” of a particular nucleic acid refer to a sequence that has the complementary nucleotide at all or substantially all positions of the nucleic acid and, therefore, specifically binds to the nucleic acid. As used herein, a “capture sequence” refers to a sequence that is the reverse complement of a sequence on a target nucleic acid, and therefore specifically binds the target nucleic acid.

A “polymerase” refers to an enzyme that performs template-directed synthesis of polynucleotides, e.g., DNA and/or RNA. The term encompasses both the full-length polypeptide and a domain that has polymerase activity. DNA polymerases are well-known to those skilled in the art, including but not limited to DNA polymerases isolated or derived from Pyrococcus furiosus, Thermococcus litoralis, and Thermotoga maritime, or modified versions thereof. Additional examples of commercially available polymerase enzymes include, but are not limited to: Klenow fragment (New England Biolabs® Inc.), Taq DNA polymerase (QIAGEN), 9° N™ DNA polymerase (New England Biolabs® Inc.), Deep Vent™ DNA polymerase (New England Biolabs® Inc.), Manta DNA polymerase (Enzymatics®), Bst DNA polymerase (New England Biolabs® Inc.), and phi29 DNA polymerase (New England Biolabs® Inc.).

Polymerases include both DNA-dependent polymerases and RNA-dependent polymerases such as reverse transcriptase. At least five families of DNA-dependent DNA polymerases are known, although most fall into families A, B and C. Other types of DNA polymerases include phage polymerases. Similarly, RNA polymerases typically include eukaryotic RNA polymerases I, II, and III, and bacterial RNA polymerases as well as phage and viral polymerases. RNA polymerases can be DNA-dependent and RNA-dependent.

As used herein, the term “partitioning” or “partitioned” refers to separating a sample into a plurality of portions, or “partitions.” Partitions are generally physical, such that a sample in one partition does not, or does not substantially, mix with a sample in an adjacent partition. Partitions can be solid or fluid. In some embodiments, a partition is a solid partition, e.g., a microchannel. In some embodiments, a partition is a fluid partition, e.g., a droplet. In some embodiments, a fluid partition (e.g., a droplet) is a mixture of immiscible fluids (e.g., water and oil). In some embodiments, a fluid partition (e.g., a droplet) is an aqueous droplet that is surrounded by an immiscible carrier fluid (e.g., oil).

As used herein, “universal adapter sequence” refers to a short heterologous nucleotide sequence, linked to a set of diverse nucleic acids, that is not specific for one sample, cell, or partition. In some embodiments, a universal adapter sequence allows for use of a common (universal) primer to amplify and/or facilitate sequencing of the set. In some embodiments, a universal adapter sequence comprises a PCR priming sequence. As used herein, a PCR priming sequence is a sequence to which a PCR primer binds.

As used herein a “barcode” is a short nucleotide sequence (e.g., at least about 4, 6, 8, 10, or 12, nucleotides long) that identifies a molecule to which it is conjugated. Barcodes can be used, e.g., to identify molecules in a partition. Such a partition-specific barcode should be unique for that partition as compared to barcodes present in other partitions. For example, partitions containing target RNA from single-cells can be subjected to reverse transcription conditions using primers that contain a different partition-specific barcode sequence in each partition, thus incorporating a copy of a unique “cellular barcode” into the reverse transcribed nucleic acids of each partition. Thus, nucleic acid from each cell can be distinguished from nucleic acid of other cells due to the unique “cellular barcode.” In some cases, the cellular barcode is provided by a “bead-specific barcode” or “bead barcode” that is present on oligonucleotides conjugated to a bead, wherein the bead-specific barcode is shared by (e.g., identical or substantially identical amongst) all, or substantially all, of the oligonucleotides conjugated to that bead. Thus, cellular and bead-specific barcodes can be present in a partition, attached to a bead, or bound to cellular nucleic acid as multiple copies of the same barcode sequence. Cellular or bead-specific barcodes of the same sequence can be identified as deriving from the same cell, partition, or bead. Such partition-specific, cellular, or bead-specific barcodes can be generated using a variety of methods, which methods result in the barcode conjugated to or incorporated into a solid or hydrogel support (e.g., a solid bead or particle or hydrogel bead or particle). In some cases, the partition-specific, cellular, or bead-specific barcode is generated using a split and mix (also referred to as split and pool) synthetic scheme as described herein. A partition-specific barcode can be a cellular barcode and/or a bead-specific barcode. Similarly, a cellular barcode can be a partition specific barcode and/or a bead-specific barcode. Additionally, a bead-specific barcode can be a cellular barcode and/or a partition-specific barcode.

In some embodiments, barcodes uniquely identify the molecule to which it is conjugated and are referred to as a unique molecular identifier (UMI). The number of nucleotides of the UMI, which can be continuous, or discontinuous, will depend on the number of UMI sequences required. In some embodiments, the number of UMIs available are many times (e.g., 2×, 10×, 100×, etc.) higher than possible conjugation partners, thereby reducing the chance of rare duplicates being linked to different molecules. In some embodiments, pools of different UMIs are present in a partition and the composition of the pool acts as an identifier for the partition, with some UMIs being in common with some other partitions but the total pool of UMIs being unique or substantially unique between partitions. UMI sequences can be generated for example as random sequences of a set length, and in some embodiments is identified by a flanking known sequence.

The length of the barcode sequence determines how many unique samples can be differentiated. For example, a 1 nucleotide barcode can differentiate 4, or fewer, different samples or molecules; a 4-nucleotide barcode can differentiate 4⁴ or 256 samples or less; a 6-nucleotide barcode can differentiate 4096 different samples or less; and an 8-nucleotide barcode can index 65,536 different samples or less. Additionally, barcodes can be attached to both strands, for example, through barcoded primers for both first and second strand synthesis.

Barcodes are typically synthesized and/or polymerized (e.g., amplified) using processes that are inherently inexact. Thus, barcodes that are meant to be uniform (e.g., a cellular, bead-specific, or partition-specific barcode shared amongst all barcoded nucleic acid of a single partition, cell, or bead) can contain various N−1 deletions or other mutations from the canonical barcode sequence. Thus, barcodes that are referred to as “identical” or “substantially identical” copies refer to barcodes that differ due to one or more errors in, e.g., synthesis, polymerization, or purification errors, and thus contain various N−1 deletions or other mutations from the canonical barcode sequence. Moreover, the random conjugation of barcode nucleotides during synthesis using e.g., a split and pool approach and/or an equal mixture of nucleotide precursor molecules as described herein, can lead to low probability events in which a barcode is not absolutely unique (e.g., different from all other barcodes of a population or different from barcodes of a different partition, cell, or bead). However, such minor variations from theoretically ideal barcodes do not interfere with the high-throughput sequencing analysis methods, compositions, and kits described herein. Therefore, as used herein, the term “unique” in the context of a bead-specific, cellular, partition-specific, or molecular barcode encompasses various inadvertent N−1 deletions and mutations from the ideal barcode sequence. In some cases, issues due to the inexact nature of barcode synthesis, polymerization, and/or amplification, are overcome by oversampling of possible barcode sequences as compared to the number of barcode sequences to be distinguished (e.g., at least about 2-, 5-, 10-fold or more possible barcode sequences). For example, 10,000 cells can be analyzed using a cellular barcode having 9 barcode nucleotides, representing 262,144 possible barcode sequences. The use of barcode technology is well known in the art, see for example Katsuyuki Shiroguchi, et al. Proc Natl Acad. Sci USA., 2012 Jan. 24; 109 (4): 1347-52; and Smith, A M et al., Nucleic Acids Research Can 11, (2010). Further methods and compositions for using barcode technology include those described in U.S. 2016/0060621.

The term “bead” refers to any solid support that can be in a partition, e.g., a small particle or other solid support. Exemplary beads can include hydrogel beads. In some cases, the hydrogel is in sol form. In some cases, the hydrogel is in gel form. An exemplary hydrogel is an agarose hydrogel. Other hydrogels include, but are not limited to, those described in, e.g., U.S. Pat. Nos. 4,438,258; 6,534,083; 8,008,476; 8,329,763; U.S. Patent Appl. Nos. 2002/0,009,591; 2013/0,022,569; 2013/0,034,592; and International Patent Publication Nos. WO/1997/030092; and WO/2001/049240.

The term “sample” refers to a biological composition, such as a cell, comprising a target nucleic acid.

The term “amplification reaction” refers to any in vitro means for multiplying the copies of a target sequence of nucleic acid in a linear or exponential manner. Such methods include but are not limited to polymerase chain reaction (PCR); DNA ligase chain reaction (see U.S. Pat. Nos. 4,683,195 and 4,683,202; PCR Protocols: A Guide to Methods and Applications (Innis et al., eds., 1990)) (LCR); QBeta RNA replicase and RNA transcription-based amplification reactions (e.g., amplification that involves T7, T3, or SP6 primed RNA polymerization), such as the transcription amplification system (TAS), nucleic acid sequence based amplification (NASBA), and self-sustained sequence replication (3SR); isothermal amplification reactions (e.g., single-primer isothermal amplification (SPIA)); as well as others known to those of skill in the art.

“Amplifying” refers to a step of submitting a solution to conditions sufficient to allow for amplification of a polynucleotide if all of the components of the reaction are intact. Components of an amplification reaction include, e.g., primers, a polynucleotide template, polymerase, nucleotides, and the like. The term “amplifying” typically refers to an “exponential” increase in target nucleic acid. However, “amplifying” as used herein can also refer to linear increases in the numbers of a select target sequence of nucleic acid, such as is obtained with cycle sequencing or linear amplification. In an exemplary embodiment, amplifying refers to PCR amplification using a first and a second amplification primer. In some embodiments, an amplification primer introduces a heterologous sequence to the amplification product. In an exemplary embodiment, the amplification primer introduces a universal adapter sequence to the amplification product to facilitate sequencing of the amplification product.

“Polymerase chain reaction” or “PCR” refers to a method whereby a specific segment or subsequence of a target double-stranded DNA, is amplified in a geometric progression. PCR is well known to those of skill in the art (see, e.g., U.S. Pat. Nos. 4,683,195 and 4,683,202; and PCR Protocols: A Guide to Methods and Applications, Innis et al., eds, 1990). Exemplary PCR reaction conditions typically comprise either two or three step cycles. Two step cycles have a denaturation step followed by a hybridization/elongation step. Three step cycles comprise a denaturation step followed by a hybridization step followed by a separate elongation step.

The term “deconvolution” refers to the assignment of 2 barcodes and the beads they were attached to as being from the same partition or originally occupying the same partition. Deconvolution can be determined by the detection of the two barcodes on a single nucleic acid fragment during sequencing.

The term “about” refers to the usual error range for the respective value that is known by a person of ordinary skill in the art for this technical field, for example, a range of ±10%, ±5%, or ±1% can encompass the recited value, even if the recited value is not modified by the term “about.”

It will be understood that any range of numerical values disclosed herein can include the endpoints of the range, and any values or sub-ranges in between the endpoints. For example, a range of 1 to 10 includes a range from 2 to 9, 3 to 8, 4 to 7, 5 to 6, 1 to 5, 2 to 5, 2 to 10, 3 to 10, and so on. The values typically include one significant digit.

### DETAILED DESCRIPTION OF THE INVENTION

### Introduction

Methods and compositions for barcoding cDNAs from single cells and nucleotide sequencing are provided. Because library preparation methods for single cell (sc) RNA-seq, like other RNA-seq methods, require reverse transcription of the RNA into cDNA, as well as additional enrichment, purification, and amplification steps (and fragmentation steps, in some methods) to yield a sequencing-ready RNA-seq cDNA library, traditional library preparation methods have the potential to result in a loss of data. This loss is particularly notable when analyzing transcripts present at only a few copies per cell. The instant disclosure provides solutions to the potential data loss by, for example, reducing the number of steps required in the library preparation. For example, a typical RNA-seq library preparation workflow may include loading cells and beads into a droplet-based single cell isolation system (e.g., ddSEQ Isolator), performing reverse transcription, and performing a cleanup and size selection step, a PCR amplification step, another cleanup step, a fragmentation and adapter addition step, and an additional cleanup step. The methods disclosed herein eliminate the need for most of these additional separate steps. Only the PCR amplification of the cDNA sample and one or more cleanup steps would be included.

In addition, in some embodiments, the methods and compositions in this disclosure also provide a solution to problems associated with more than one barcoded bead being present in a single partition. Beads conjugated to oligonucleotides are used in RNA-seq applications having many different partitions, such as droplets. Barcodes can be delivered to partitions, such as droplets, using beads as the delivery vehicle. The beads can be used to deliver many copies of an oligonucleotide to a partition, and the oligonucleotide may have barcode sequences that are unique for the bead to which the oligonucleotide is linked. In-partition reverse transcription of cellular RNAs, using the oligonucleotides, and in-partition strand synthesis are used to generate a plurality of copies of a bead-specific barcode sequence linked to a sample cDNA. Tagging cDNA with a barcode sequence in partitions can provide important information through the sequencing and analysis of the barcode sequences along with the tagged cDNA molecules. However, a number of partitions may have more than one bead, and therefore, some partitioned samples may be labeled by more than one barcode sequence. Linkage of two different bead-specific barcodes to a sample nucleic acid can be detected using sequencing to determine whether and which bead-specific barcodes originated in the same partition. Notably, the presence of more than one bead-specific barcoded oligonucleotide (e.g., two different bead-specific-barcoded primers linked to different beads in the same partition) can interfere with sequence analysis and quantification because different barcodes are assumed to be from different partitions when in fact some fraction of the barcodes occur together (for example as a function of a Poisson distribution). Some of the disclosed methods enable deconvolution (i.e., determination that multiple bead barcodes are from the same partition and accounting for that in the sequencing analysis) and appropriate use of the data from such combinations or disregarding (e.g., discarding) data from such partitions, leaving the remaining data with reduced background.

A. Methods and Compositions of Barcoding cDNAs Using an Untailed Approach

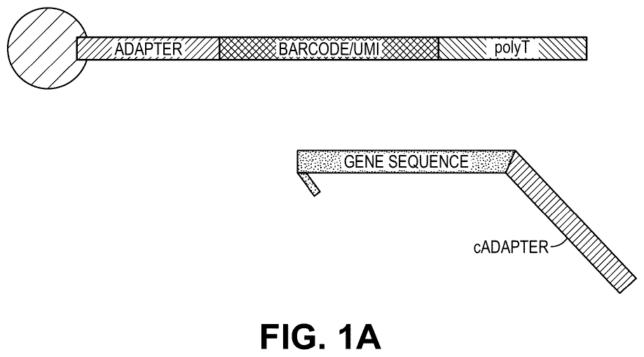

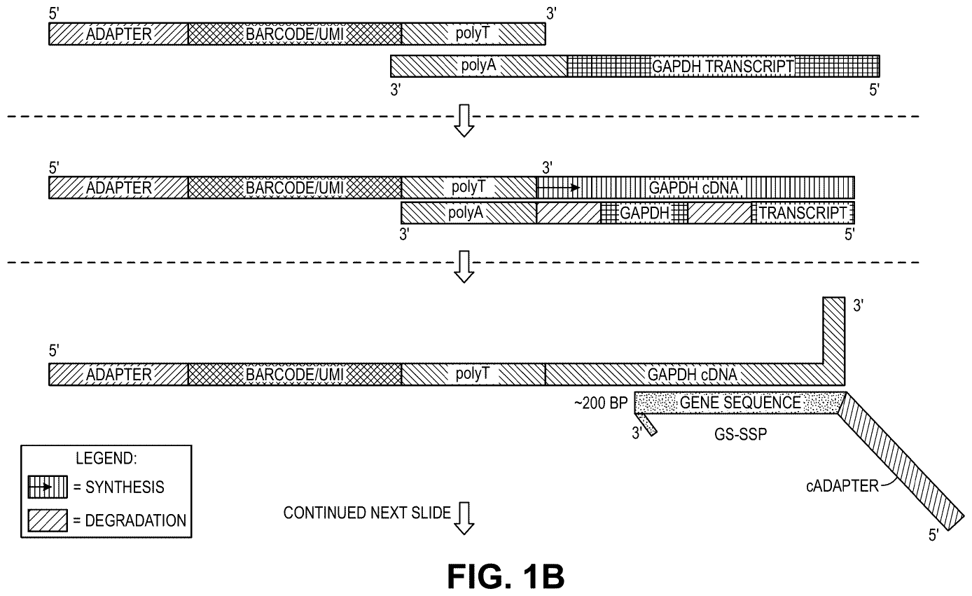

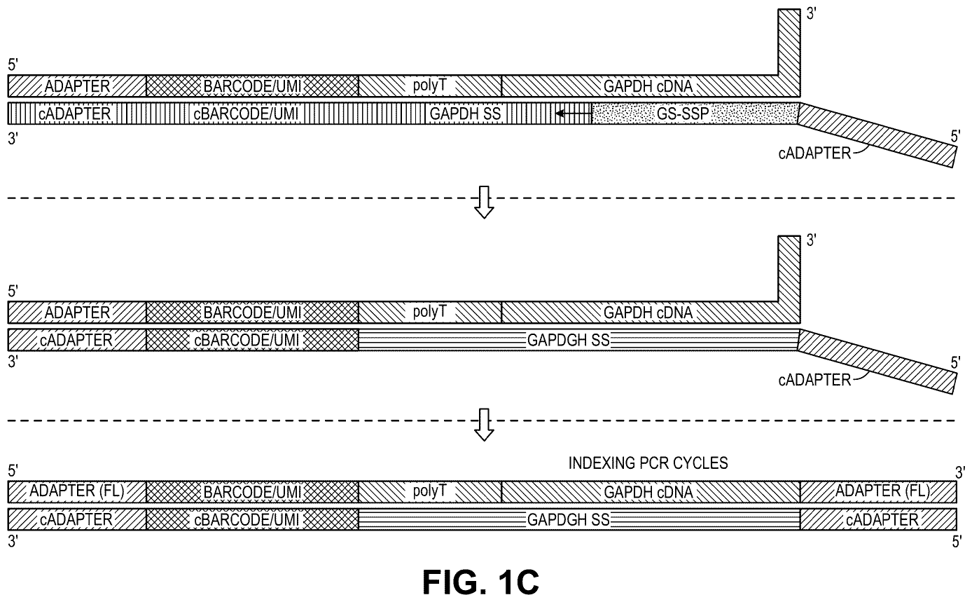

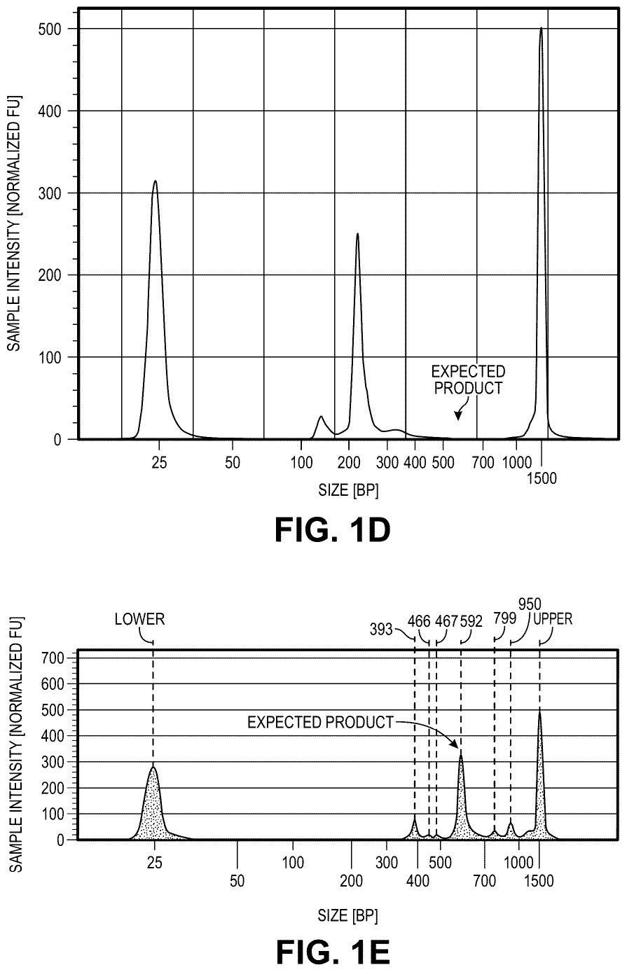

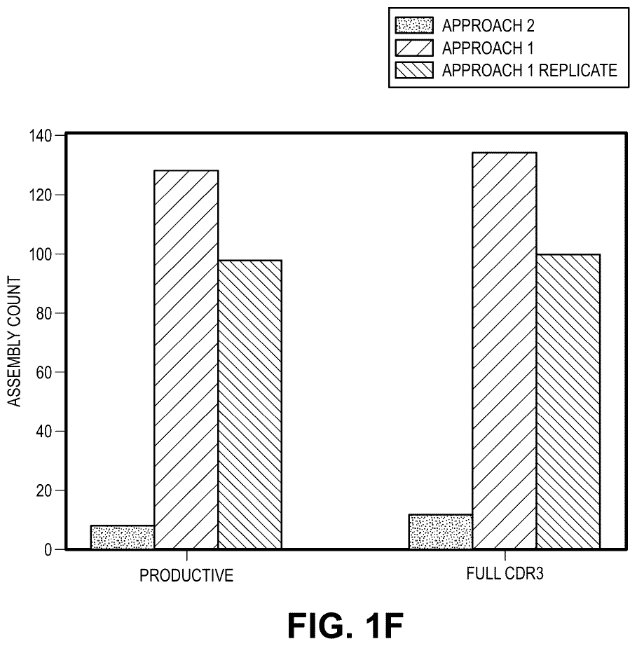

In some embodiments, the disclosure provides methods of barcoding cDNAs from cells in a partition using an untailed approach. In these embodiments, neither the barcoding oligonucleotide nor the gene-specific detection oligonucleotide include a tail sequence that will be incorporated in the final product for sequencing. These embodiments produce a final product that is a double-stranded DNA product for sequencing that includes a universal adapter sequence at both ends of the molecule to facilitate sequencing. The final product also includes a bead-specific barcode and a portion of the target gene that has a length suitable for sequencing (see, e.g., final product in FIG. 1C). In some embodiments, the portion of the target gene is about 50 and 600 nucleotides. Therefore, the portion of the target gene in the final product is between about 50, 60, 70, 80, 90, 100, 150, 200, 250, 300, 350, 400, 450, 500, 550, or 600 nucleotides, or any length in between. In certain embodiments, the portion of the target gene in the final product is about 180 to 220 nucleotides.

In some embodiments, the methods include a step of providing a plurality of partitions (e.g., droplets in an emulsion or microwells). In some embodiments, different partitions include at least one bead (e.g., a hydrogel bead) linked to a plurality of copies of a barcoding oligonucleotide; and a single cell, single nucleus, or nucleic acids from a single cell or nucleus. In other embodiments, the partitions also include a plurality of gene-specific detection oligonucleotides; a reverse transcriptase; a DNA polymerase; or a combination thereof.

In some embodiments, the cell is obtained from a biological sample. Biological samples can be obtained from any biological organism, e.g., an animal, plant, fungus, pathogen (e.g., bacteria or virus), or any other organism. In some embodiments, the biological sample is from an animal, e.g., a mammal (e.g., a human or a non-human primate, a cow, horse, pig, sheep, cat, dog, mouse, or rat), a bird (e.g., chicken), or a fish. A biological sample can be any tissue or bodily fluid obtained from the biological organism, e.g., blood, a blood fraction, or a blood product (e.g., serum, plasma, platelets, red blood cells, and the like), sputum or saliva, tissue (e.g., kidney, lung, liver, heart, brain, nervous tissue, thyroid, eye, skeletal muscle, cartilage, or bone tissue); cultured cells, e.g., primary cultures, explants, and transformed cells, stem cells, stool, urine, etc. In some embodiments, the sample is a single-cell sample. In some embodiments, the cells are prokaryotic cells. In some embodiments, the cells are eukaryotic cells. In some embodiments, the cell is a mammalian cell.

In some embodiments, the barcoding oligonucleotide includes 5′-3′: a first PCR priming sequence, a bead-specific barcode sequence, and a capture sequence. The first PCR priming sequence is a short heterologous nucleotide sequence that is not specific for one sample, cell, or partition. In some embodiments, the PCR priming sequence is bound by a PCR primer in an amplification reaction.

In some embodiments of the disclosed methods, the barcoding oligonucleotide includes a capture sequence that comprises a poly T sequence (also referred to as an oligo dT sequence) for the capture of mRNA sequences from cell in the partition. This barcoding oligonucleotide also may be referred to as an mRNA capture oligonucleotide as it may bind to the poly A tail sequence of mRNAs. In some embodiments, the poly T is a single stranded sequence of deoxythymine (dT). The length of the poly T sequence can vary, for example, from about 6 bases to about 40 bases, or any number within that range, and may be a mixture of lengths. For example, in some embodiments, the capture sequence comprises a poly T sequence of about 6, 10, 15, 20, 25, 30, 35, or 40 Ts. In certain embodiments, the capture sequence comprises 22-36 Ts. In other embodiments of the disclosed methods, the barcoding oligonucleotide or RNA capture oligonucleotide includes a capture sequence that is a DNA sequence that is reverse complementary to a portion of the target RNA (e.g., the capture sequence may comprise a portion of a target gene sequence to capture a corresponding target mRNA).

In some embodiments of the disclosed methods, the gene-specific detection oligonucleotide includes 5′-3′: a second PCR priming sequence and a reverse complement sequence of a portion of a target gene from the cell or nucleus in the partition. In some embodiments, the gene-specific detection oligonucleotide may be referred to as a gene-specific second strand primer. In some embodiments, the portion of the target gene in the gene-specific detection oligonucleotide may bind to a sequence that is located between about 50 and 600 nucleotides upstream of the capture sequence or the DNA equivalent thereof. Therefore, the gene-specific detection oligonucleotide may bind to a sequence that is located between about 50, 60, 70, 80, 90, 100, 150, 200, 250, 300, 350, 400, 450, 500, 550, or 600, nucleotides upstream of the capture sequence, or any length in between. In certain embodiments, the gene-specific detection oligonucleotide binds to a sequence that is 180 to 220 nucleotides upstream of the capture sequence (see, e.g., bottom panel of FIG. 1B).

In some embodiments, the plurality of gene-specific detection oligonucleotides includes at least 10, 50, 100, 500, or 1000 gene-specific detection oligonucleotides specific for different mRNAs present in the single cell. In other embodiments, the plurality includes gene-specific detection oligonucleotides are specific for all or substantially all of the cell transcriptome. In other embodiments, the gene-specific detection oligonucleotides target a smaller subset of the transcriptome. In certain embodiments, at least one gene-specific detection oligonucleotide of the plurality of gene-specific detection oligonucleotides is specific for a target gene that is known to be highly or regularly expressed in the single cell or nucleus. In some embodiments, the target gene may be a “housekeeping” gene (e.g., gene required for basis and ubiquitous cellular functions). For example, in certain embodiments, the housekeeping gene is glyceraldehyde 3-phosphate dehydrogenase (GAPDH).

In some embodiments, the step of providing a plurality of partitions includes providing intact cells in partitions and subsequently lysing the cells in the partitions. The cells may be lysed using any method known in the art. For example, in certain embodiments, the cells are lysed with a buffer containing a detergent.

In some embodiments, the methods include performing several steps of the library preparation within the partitions (See, e.g., two or more of the steps of FIG. 1B to middle panel of FIG. 1C). In other embodiments, only RNA capture (annealing sequences of cellular RNA to some copies of the barcoding oligonucleotides) is performed within the partitions. In some embodiments, the steps performed in the partitions include releasing the barcoding oligonucleotides from the beads; performing reverse transcription by annealing cellular RNAs from the cell or nucleus to some copies of the barcoding oligonucleotides and forming first strand cDNAs by extending the barcoding oligonucleotides with the reverse transcriptase using the cellular RNAs as a template, thereby creating RNA:DNA duplexes, wherein the first strand cDNAs comprise the first PCR priming sequence, the bead-specific barcode sequence, the capture sequence, and a portion of the target gene sequence (top and middle panels of FIG. 1B); degrading the RNA in the RNA:DNA duplexes (middle panel of FIG. 1B) by removing at least a portion of the RNA in the duplex; and performing second strand synthesis to form second strand cDNAs using first strand cDNAs as templates and the gene-specific detection oligonucleotides as primers, thereby forming double-stranded cDNAs from a plurality of different RNAs (bottom panel of FIG. 1B to middle panel of FIG. 1C). The analysis of a library obtained with the disclosed methods is described further below in Example 3 (see FIGS. 1D-IF).

The surface of the bead can be modified to include a linker for attaching barcode oligonucleotides. The linkers may comprise a cleavable moiety, which may be cleaved in the partitions. Non-limiting examples of cleavable moieties include a disulfide bond, a dioxyuridine moiety, and a restriction enzyme recognition site. The cleavable sequence can be any cleavable sequence that can be targeted enzymatically or otherwise while leaving the rest of the nucleic sequences in the mixture intact. In some embodiments, the cleavable sequence comprises one or more uracils. For example, the cleavable sequence can include 1, 2, 3, 4, or more uracils, which can be contiguous. Uracils can be selectively removed, and the backbone cleaved (nicked), by contacting with uracil DNA glycosylase and endonuclease VIII, which excises the one or more uracil. Uracil DNA glycosylase and endonuclease VIII is available commercially, for example from New England Biolabs as “USER™” (Uracil-Specific Excision Reagent). In some embodiments, the cleavable sequence comprises one or more ribonucleotide(s). For example, the cleavable sequence can include 1, 2, 3, 4 or more ribonucleotides, which can be contiguous. This allows one to use an enzyme that selectively cleaves ribonucleotides and does not substantially cleave deoxyribonucleotides. For example, in some embodiments, RNAse H is used to specifically cleave at a ribonucleotide in the cleavable sequence. In some embodiments, the cleavable sequence comprises a restriction enzyme recognition or cleavage site (collectively referred to as a “restriction site”) located between the first oligonucleotide and the second oligonucleotide. In these embodiments, the long oligonucleotide can be cleaved with a restriction enzyme that cleaves the restriction site on the long oligonucleotide without cleaving the linking oligonucleotide. Examples of such enzymes nicking endonuclease. Preferably, the restriction enzyme is selected such that its recognition and/or cleavage site only occurs in the cleavable sequence and does not occur elsewhere in the oligonucleotides in the mixture. In some other embodiments, the bead-specific barcoding oligonucleotides are released by dissolving the bead.

Reverse transcription can be performed using any suitable reverse transcriptases, such as, but not limited to Maxima RNase⁺ (Thermo), Maxima RNase (Thermo), murine leukemia virus (MLV) reverse transcriptase (Gerard and Grandgenett, Journal of Virology 15:785-797, 1975; Verma, Journal of Virology 15:843-854, 1975) or SEQ ID NO: 1, feline leukemia virus (FLV) reverse transcriptase (Rho and Gallo, Cancer Lett., 10:207-221, 1980 or SEQ ID NO:2, bovine leukemia virus (BLV) (Demirhan et al., Anticancer Res., 16:2501-5, 1996; Drescher et al., Arch Geschwulstforsch., 49:569-79, 1979), Avian Myeloblastosis Virus (AMV) reverse transcriptase, Respiratory Syncytial Virus (RSV) reverse transcriptase, Equine Infectious Anemia Virus (EIAV) reverse transcriptase, Rous-associated Virus-2 (RAV2) reverse transcriptase, SUPERSCRIPT II reverse transcriptase, SUPERSCRIPT III reverse transcriptase (U.S. Pat. Nos. 8,541,219, 7,056,716, 7,078,208), THERMOSCRIPT reverse transcriptase and MMLV RNase H⁻ reverse transcriptase and Sensiscript (Qiagen).

In some embodiments, the RNA in the RNA:DNA heteroduplex is degraded by an enzyme having RNase H⁺ activity. Ribonuclease H (RNase H or RNH) refers to a family of non-sequence-specific endonuclease enzymes that cleave RNA in an RNA/DNA heteroduplex. These enzymes cleave RNA backbone phosphodiester bonds to leave a 3′ hydroxyl and a 5′ phosphate group. In some embodiments of the disclosed methods, the reverse transcription and the RNA:DNA duplex degradation are performed by a single enzyme having RNase H⁺ activity. In some embodiments, the enzyme is Maxima Reverse Transcriptase (RNase H⁺) (Thermo), M-MLV RT (Thermo), or iScript (Bio-Rad).

In some embodiments, after performing strand synthesis, the methods can include inactivating the polymerase and reverse transcriptase in the partitions. In certain embodiments, the inactivating comprises applying heat to the partitions and raising the temperature sufficiently high to inactivate the enzymes. In some embodiments, the inactivating comprises incubating the partitions at 75-90 degrees Celsius.

In some embodiments, the methods include performing any remaining steps outside the individual partitions (see, e.g., bottom panel of FIG. 1C). In some embodiments, these steps include generating a bulk mixture by combining contents of the partitions; and performing an amplification (e.g., PCR) reaction with primers that bind to the first or second PCR priming sequence or reverse complement sequence. In some embodiments, the amplification reaction is performed with primers that introduce heterologous sequences. For example, in some embodiments, the amplification reaction is performed with a first primer comprising a first universal adapter sequence and a bead-specific barcode sequence or reverse complement sequence, and a second primer that comprises a second universal adapter sequence and a target gene sequence or reverse complement sequence. The resulting double stranded sequencing cDNAs include 5′-3′: the first universal adapter sequence, the bead-specific barcode sequence, the target gene sequence, and the second universal adapter sequence.

Universal adapter sequences are sequences that are used to end-label nucleic acids from cells in a partition in each of two or more partitions for use, for example, in sequencing applications known in the art. In some embodiments, the universal adapter sequence may be referred to as a sequencing adapter sequence when the adapter sequence facilitates sequencing methods. In some embodiments, the cDNAs from two or more partitions are end-labelled for use with Illumina, Ion Torrent, Element Biosciences, or BGI sequencing technology. Any known adapter sequences may be suitable for use as the universal adapter sequence in the disclosed methods. For example, in some embodiments, the universal adapters may comprise a P5 adapter sequence (5′ AAT GAT ACT GCG ACC GA 3′ (SEQ ID NO: 3)), a P7 adapter sequence (5′ CAA GCA GAA GAC GGC ATA CGA GAT 3′ (SEQ ID NO: 4)) (Illumina), an Ion Torrent P1 adapter sequence (5′ CCA CTA CGC CTC CGC TTT CCT CTC TAT GGG CAG TCG GTG AT 3′ (SEQ ID NO:5)), an Ion Torrent A adapter sequence (5′ CCA TCT CAT CCC TGC GTG TCT CCG ACT CAG 3′ (SEQ ID NO: 6)), an Element Adept surface primer sequence, or a BGI adapter sequence. In some embodiments, the primers used in the amplification reaction include an index sequence, thereby forming double stranded sequencing cDNAs comprising the index sequence.

Also provided are a plurality of partitions (e.g., droplets in emulsion or microwells). In some embodiments, the different partitions include at least one bead (e.g., a hydrogel bead) linked to a plurality of copies of a barcoding oligonucleotide having 5′-3′: a first PCR priming sequence, a bead-specific barcode sequence, and a capture sequence; a plurality of gene-specific detection oligonucleotides, wherein each gene-specific detection oligonucleotide comprises 5′-3′: a second PCR priming sequence and a portion of a target gene reverse complement sequence wherein the portion is 50-600 nucleotides away from the capture sequence or the DNA equivalent thereof; a reverse transcriptase; a polymerase; and a single cell (e.g., a mammalian cell), single nucleus, or nucleic acids from a single cell or nucleus.

B. Methods and Compositions of Barcoding cDNAs Using a Single Tailed Approach

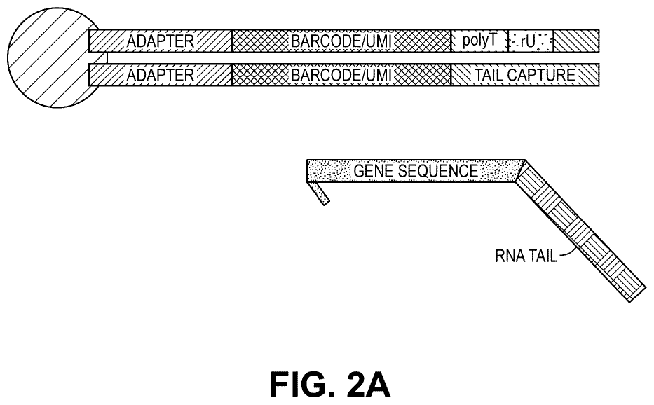

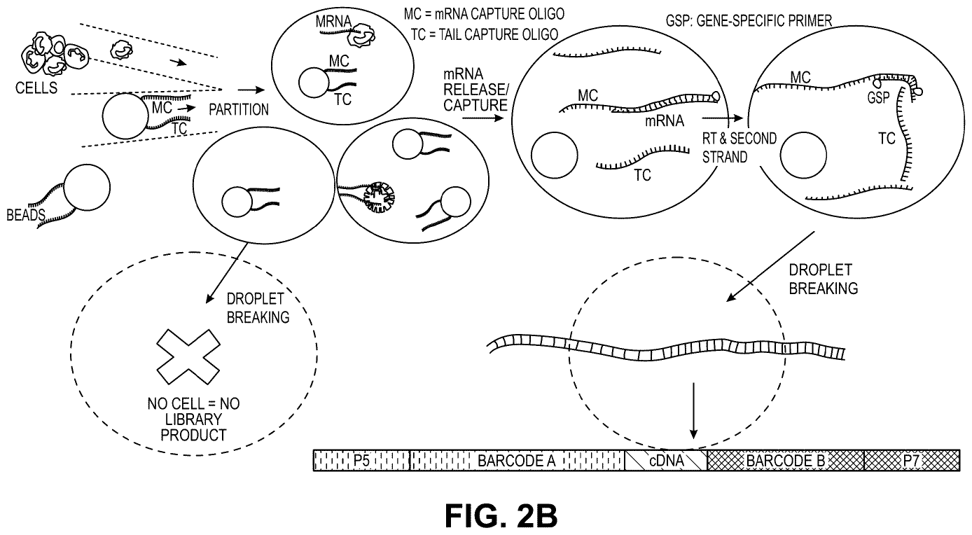

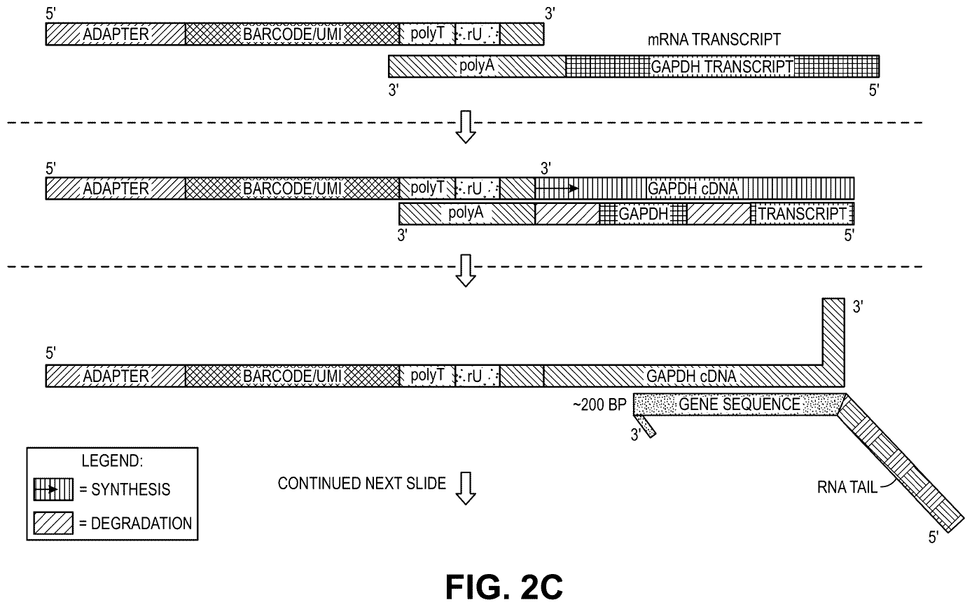

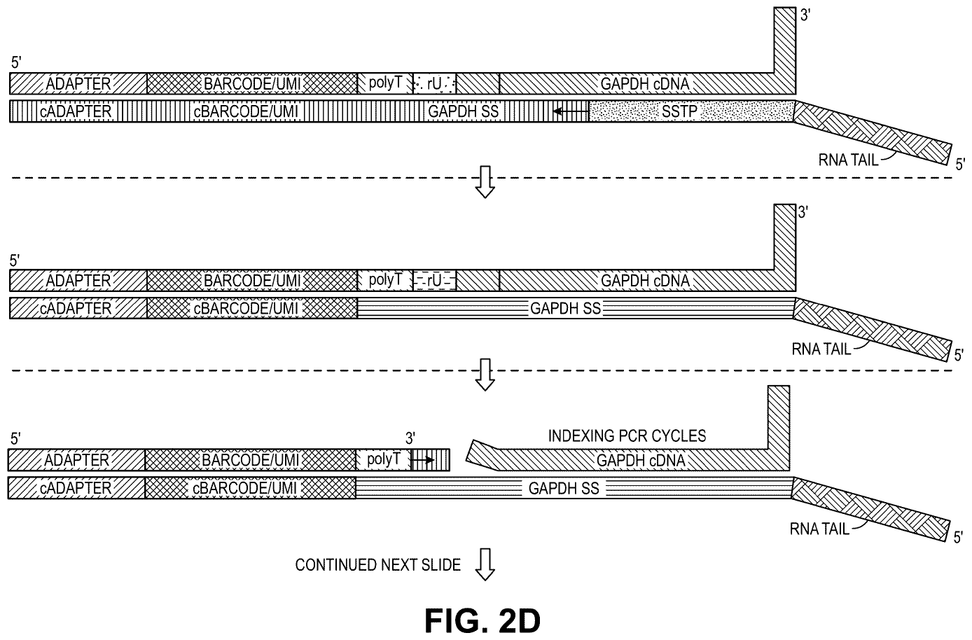

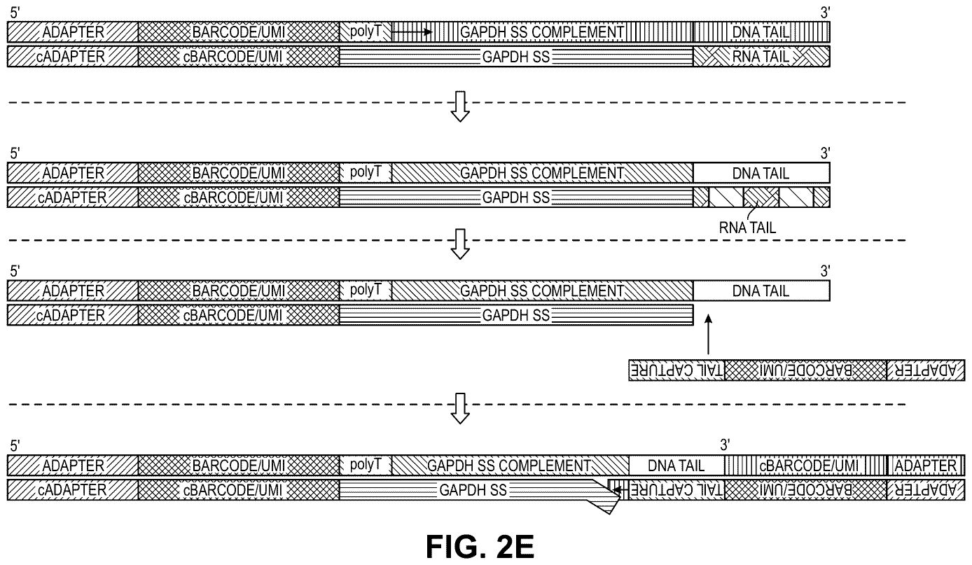

Also provided are methods of barcoding cDNAs from cells in a partition using a single tailed approach. In these embodiments, the gene-specific detection oligonucleotide includes a tail sequence that is incorporated in the final product for sequencing. These methods produce a final product that is a double-stranded DNA product for sequencing that includes a universal adapter sequence at both ends of the molecule. The final product also includes a first bead-specific barcode sequence, a capture sequence, a portion of the target gene that has a length suitable for sequencing, and a second bead-specific barcode sequence (see, e.g., bottom panel in FIG. 2E). The description of various components and steps described for the methods in Section A. above (e.g., partitions, beads, cells, universal adapter sequences, reverse transcription, RNA degradation, length of portion of target gene sequence present in the final product for sequencing) also applies to the methods described in this section unless otherwise noted in this section.

In some embodiments of the disclosed methods, the bead is linked to a plurality of copies of a first barcoding oligonucleotide and a plurality of copies of a second barcoding oligonucleotide (see, e.g., FIG. 2A). In some embodiments, the first barcoding oligonucleotide includes 5′-3′: a first PCR priming sequence, a bead-specific barcode sequence, a capture sequence, and a site labile to a single stranded break or nick. In some embodiments, the capture sequence is a poly T sequence. In some embodiments, the site labile to a single stranded break or nick comprises at least one RNA nucleotide. In some embodiments, the at least one RNA nucleotide is near the beginning or the middle of the capture sequence. In some embodiments, RNase H⁺ activity produces a single stranded break at the site. In certain embodiments, the site labile to a single stranded break comprises at least four RNA bases. In some embodiments, the site comprises at least four uracil nucleotides in a poly T sequence. In other embodiments, heating in the presence of divalent cations produces a single stranded break at the site. In certain embodiments, the site labile to a single stranded break or nick comprises at least one uracil nucleotide in a poly T sequence.

In some embodiments, the first barcoding oligonucleotide may be referred to as an RNA capture oligonucleotide as the poly T sequence or other capture sequence binds to RNAs. In embodiments where the capture sequence is a poly T sequence, the length of the poly T sequence can vary, for example, from about 6 bases to about 40 bases, or any number within that range, and may be a mixture of lengths. For example, in some embodiments, the capture sequence comprises a poly T sequence of about 6, 10, 15, 20, 25, 30, 35, or 40 Ts. In certain embodiments, the capture sequence comprises 22-36 Ts. In other embodiments of the disclosed methods, the barcoding oligonucleotide or RNA capture oligonucleotide includes a capture sequence that is a reverse complement sequence of a portion of the target RNA sequence (e.g., the capture sequence may comprise a portion of a target gene sequence that is the reverse complement of a corresponding mRNA sequence).

In some embodiments, the second barcoding oligonucleotide includes 5′-3′: a second PCR priming sequence, the bead-specific barcode sequence, and a tail capture sequence. In some embodiments, the second barcoding oligonucleotide may be referred to as a tail capture oligonucleotide as it includes a DNA sequence that is the same sequence as the cleavable tail sequence of the gene-specific detection oligonucleotide (which also may be referred to as a gene-specific second strand tailed primer), and it does not include any RNA nucleotides. The length of the tail capture sequence can vary, for example, from about 6 bases to about 50 bases and may be a mixture of lengths that are sufficient to bind to the tail sequence added to the cDNA. Therefore, the length of the tail capture sequence may be about 6, 10, 15, 20, 25, 30, 35, 40, 45, and 50 bases in length, or any length in between.

In some embodiments, the cleavable-tailed gene-specific detection oligonucleotides include 5′-3′: a cleavable tail sequence and a portion of a target gene DNA reverse complement sequence that binds to the cDNA 50-600 bp away from the capture sequence. Unless specified as RNA, all portions of the oligonucleotide are DNA. In some embodiments, the cleavable tail comprises a cleavable moiety as described above with respect to linkers for attaching barcode oligonucleotides to the beads. In some embodiments, the cleavable tail sequence includes an RNA tail sequence (a tail sequence that includes a plurality of RNA nucleotides). In some embodiments, the tail sequence comprises at least two, three, or four RNA nucleotides. In certain embodiments, the RNA nucleotides are located in consecutive positions in the sequence. In other embodiments, the RNA nucleotides are located in non-consecutive positions in the sequence. In some embodiments, the RNA tail sequence includes all RNA bases. The length of the RNA tail sequence can vary, for example, from about 6 bases to about 50 bases. Therefore, the length of the RNA tail sequence may be about 6, 10, 15, 20, 25, 30, 35, 40, 45, and 50 in length, or any length in between.

In some embodiments, the plurality of gene-specific detection oligonucleotides includes at least 10, 50, 100, 500, or 1000 gene-specific detection oligonucleotides specific for different mRNAs present in the single cell. In other embodiments, the plurality includes gene-specific detection oligonucleotides are specific for all or substantially all of the cell transcriptome. In other embodiments, the gene-specific detection oligonucleotides target a smaller subset of the transcriptome. In certain embodiments, at least one gene-specific detection oligonucleotide of the plurality of gene-specific detection oligonucleotides is specific for a target gene that is known to be highly or regularly expressed in the single cell. In some embodiments, the target gene is a housekeeping gene. In certain embodiments, at least one gene-specific detection oligonucleotide binds to a sequence that is 50 to 600 nucleotides upstream of the mRNA poly A tail. Therefore, the gene-specific detection oligonucleotide may bind to a sequence that is located between about 50, 60, 70, 80, 90, 100, 150, 200, 250, 300, 350, 400, 450, 500, 550, or 600, nucleotides upstream of the capture sequence, or any length in between. In some embodiments, at least one gene-specific detection oligonucleotide binds to a sequence that is 100 to 400 nucleotides base pairs upstream of the capture sequence. In some embodiments, at least one gene-specific detection oligonucleotide binds to a sequence that is 180 to 220 nucleotides base pairs upstream of the capture sequence.

In some embodiments, the methods include performing several steps of the library preparation within the partitions (see, e.g., FIGS. 2B-2E). In some embodiments, these steps include releasing the barcoding oligonucleotides from the beads; performing reverse transcription, wherein the performing comprises annealing sequences of the cellular RNAs to some copies of the first barcoding oligonucleotides and forming first strand cDNAs by extending the first barcoding oligonucleotides with the reverse transcriptase using the cellular RNAs as a template, thereby creating RNA:DNA duplexes, wherein the first strand cDNAs comprise the first PCR priming sequence, the bead-specific barcode sequence, the capture sequence, the site labile to a single stranded break or nick, and the target gene sequence (top and middle panels of FIG. 2C); degrading the RNA in the RNA:DNA duplexes (middle panel of FIG. 2C); performing a second strand synthesis to form tailed second strand gene-specific cDNAs using first strand cDNAs as templates and the gene-specific detection oligonucleotides as primers, thereby forming double-stranded cDNAs wherein the cleavable-tailed second strand gene-specific cDNAs comprise 5′-3′: the cleavable tail sequence, the target gene reverse complement sequence, the bead-specific barcode reverse complement sequence, and the first PCR priming reverse complement sequence (bottom panel of FIG. 2C to top panel of FIG. 2D); producing a single stranded break at the labile site, thereby creating a free 3′ hydroxyl group at the site (middle panel of FIG. 2D); performing a strand synthesis with a displacing polymerase to form DNA-tailed first strand cDNAs using the second strand gene-specific cDNAs as templates and the free 3′ hydroxyl group to begin extension, thereby forming tailed double-stranded cDNAs, wherein the DNA-tailed first strand cDNAs comprise 5′-3′: the first PCR priming sequence, the bead-specific barcode sequence, the target gene sequence, and a DNA tail sequence that is reverse complementary to the cleavable tail sequence (bottom panel of FIG. 2D to top panel of FIG. 2E); cleaving the cleavable tail sequence in the double stranded cDNAs, thereby producing a double-stranded cDNA with a single-stranded DNA tail on the 3′ end of the first strand (second panel from the top of FIG. 2E); annealing the DNA tail of the double-stranded cDNA to the second barcoding oligonucleotide and performing a strand synthesis using the second barcoding oligonucleotide as templates and the single-stranded DNA tail of the first strand cDNA to begin extension (bottom two panels of FIG. 2E); and performing a strand synthesis with a displacing polymerase to form double-stranded cDNAs comprising the first PCR priming sequence, the bead-specific barcode sequence, truncated capture sequence, the target gene sequence, the DNA tail sequence, the bead-specific barcode reverse complement sequence, and the second PCR priming reverse complement sequence (bottom panel of FIG. 2E).

In some embodiments, the methods include performing the remaining steps outside the individual partitions. These steps include generating a bulk mixture by combining contents of the partitions; and performing an amplification (e.g., a PCR reaction) with primers that bind to the first or second PCR priming sequence or reverse complement sequence. In some embodiments, the amplification reaction is performed with primers that introduce heterologous sequences, such as a universal or sequencing adapter sequence. In some embodiments, the resulting double stranded sequencing cDNAs include the PCR priming sequence, the bead-specific barcode sequence, the capture sequence, the target gene sequence, the DNA tail sequence, the bead-specific barcode reverse complement sequence, and the PCR priming reverse complement sequence. In some embodiments, the primers used in the amplification reaction include an index sequence, thereby forming double stranded sequencing cDNAs comprising the index sequence.

In some embodiments, after performing the PCR reaction, the methods further include determining the nucleotide sequence of the double-stranded sequencing cDNAs and performing bead deconvolution. If two different bead-specific barcode sequences are present on the same cDNA, then sequencing reads comprising either of the two bead-specific barcode sequences are from the same partition.

Also provided are pluralities of partitions (e.g., droplets in an emulsion or microwells) for use in this single tailed approach for barcoding cDNAs. In some embodiments, the different partitions include at least one bead (e.g., a hydrogel bead) linked to a plurality of copies of a first barcoding oligonucleotide and a plurality of copies of a second barcoding oligonucleotide; wherein the first barcoding oligonucleotide comprises: a first PCR priming sequence, a bead-specific barcode sequence, a capture sequence, and a site labile to a single stranded break or nick; and wherein the second barcoding oligonucleotide comprises 5′-3′: a second PCR priming sequence, a bead-specific barcode sequence, and a tail capture sequence; a plurality of cleavable-tailed gene-specific detection oligonucleotides, wherein each gene-specific detection oligonucleotide comprises: a cleavable tail sequence and a portion of a target gene DNA reverse complement sequence that binds to the cDNA 50-600 bp away from the capture sequence; a reverse transcriptase; and a single cell, nucleus, or nucleic acids from a single cell or nucleus.

C. Methods and Compositions of Barcoding cDNAs Using a Double-Tailed Approach

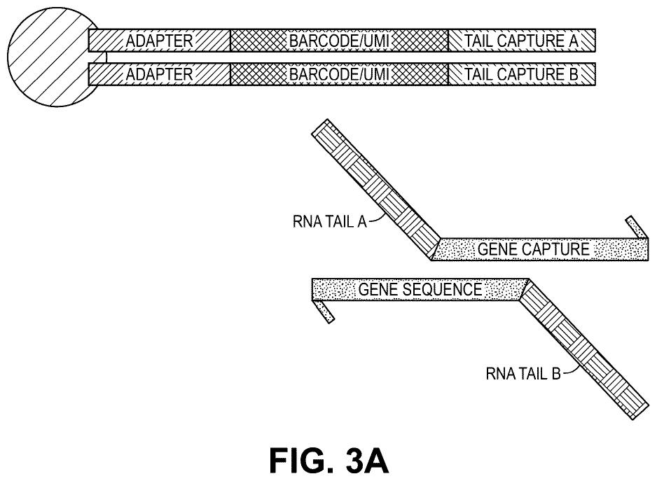

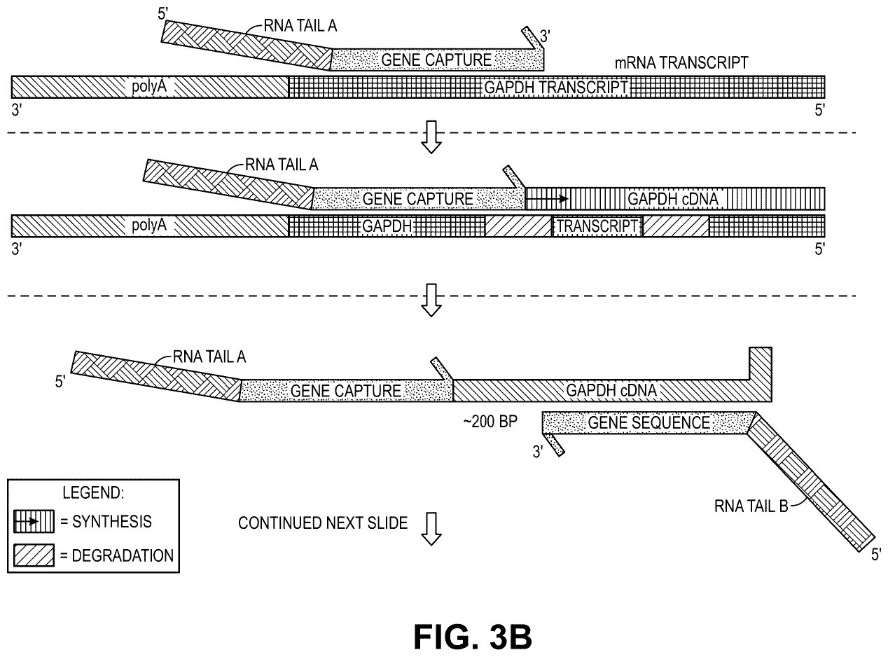

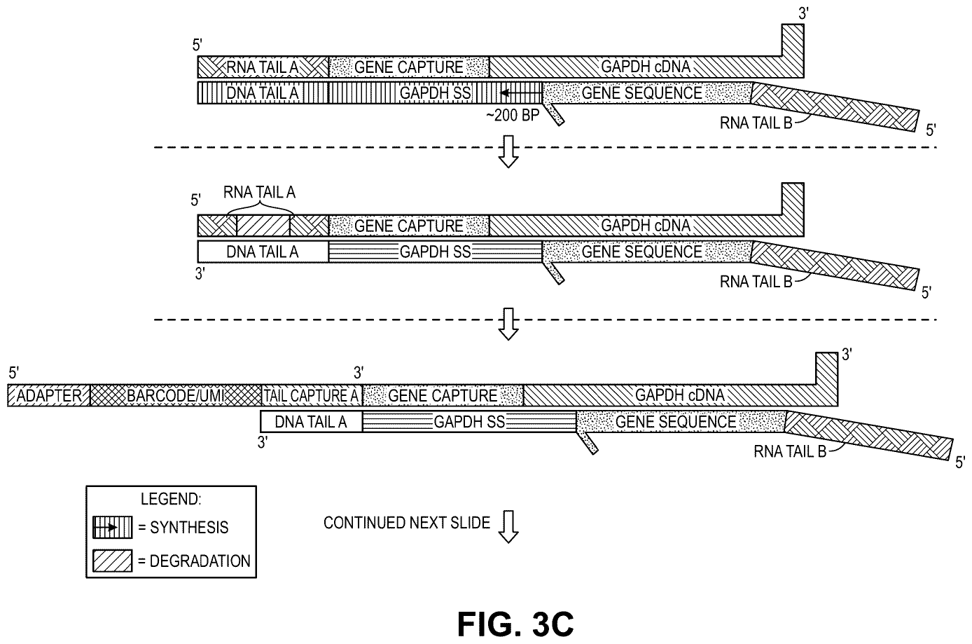

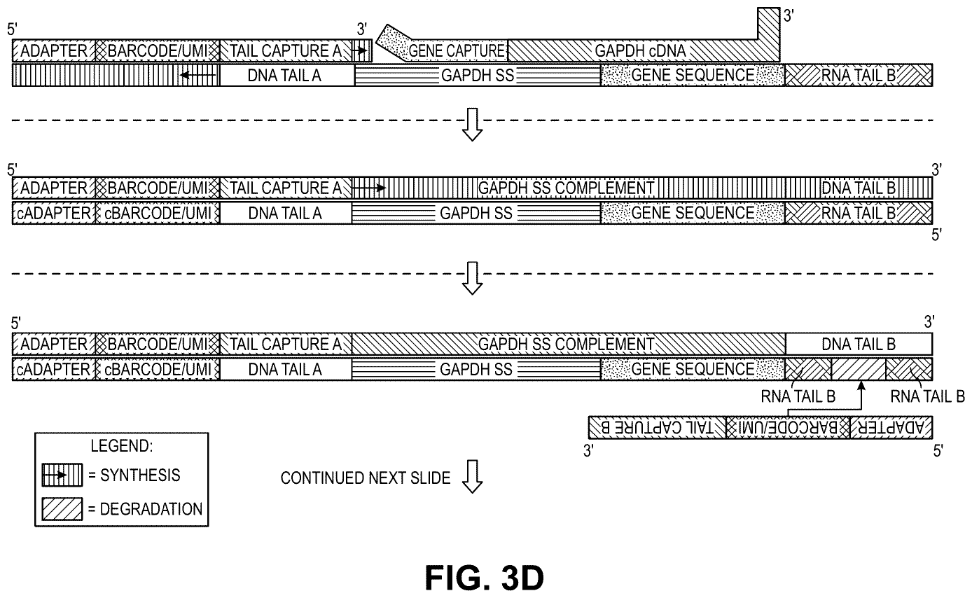

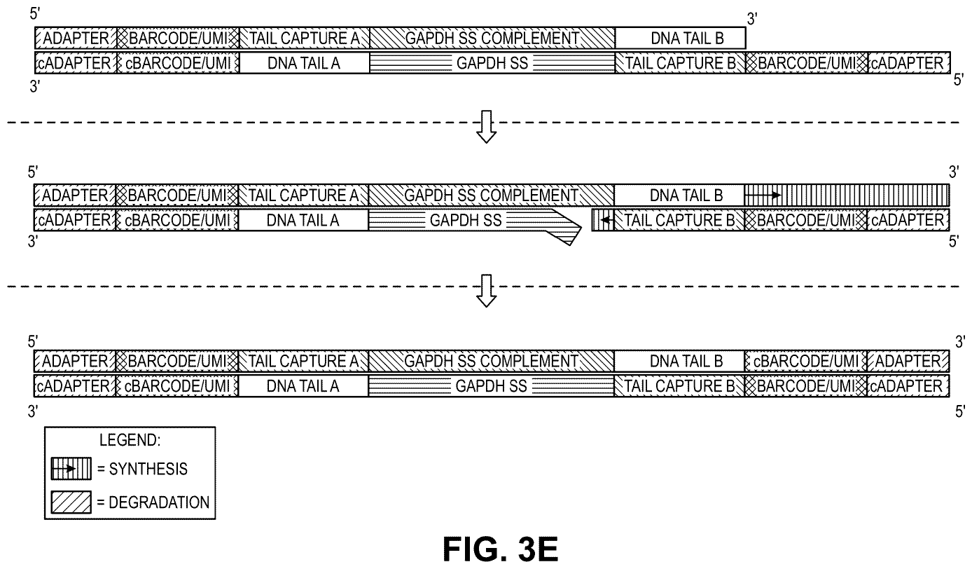

The disclosure also provides methods of barcoding cDNAs from cells in a partition using a double tailed approach. In these embodiments, pairs of gene-specific detection oligonucleotides are used for each target gene. The first gene-specific detection oligonucleotide includes a first tail sequence that is incorporated in the final product for sequencing, and the second gene-specific detection oligonucleotide includes a second tail sequence that also is incorporated in the final product for sequencing. These methods produce a final product that is a double-stranded DNA product for sequencing that includes a universal adapter sequence at both ends of the molecule. The final product also includes a first bead-specific barcode, a first tail reverse complement sequence, a reverse complement sequence of a portion of the target gene that has a length suitable for sequencing, a second tail sequence, and a second bead-specific barcode sequence (see, e.g., bottom panel in FIG. 3E). The description of various components and steps described for the methods in Sections A. and B. above (e.g., partitions, beads, cells, universal adapter sequences, reverse transcription, RNA degradation, number and type of target genes, tail sequence, length of portion of target gene sequence present in the final product for sequencing) also applies to the methods described in this section unless otherwise noted in this section.

In some embodiments of the disclosed methods, the bead is linked to a plurality of copies of a first barcoding oligonucleotide and a plurality of copies of a second barcoding oligonucleotide (FIG. 3A). In some embodiments, the first barcoding oligonucleotide includes 5′-3′: a first PCR priming sequence, a bead-specific barcode sequence, and a first tail capture sequence, and the second barcoding oligonucleotide includes 5′-3′: a second PCR priming sequence, the bead-specific barcode sequence, and a second tail capture sequence. In some embodiments, the pairs of cleavable-tailed gene-specific detection oligonucleotides include a first and second gene-specific detection oligonucleotide (FIG. 3A). In some embodiments, the first cleavable tail sequence, the second cleavable tail sequence, or both comprise a plurality of RNA nucleotides. In certain embodiments, the first gene-specific detection oligonucleotide includes 5′-3′: a first cleavable tail sequence (e.g., RNA tail A) and a target gene sequence, and the second gene-specific detection oligonucleotide includes 5′-3′: a second cleavable tail sequence (e.g., RNA tail B) and a target gene reverse complement sequence. The first gene-specific detection oligonucleotide also may be referred to as a gene-specific first strand tailed capture oligonucleotide, and the second gene-specific detection oligonucleotide also may be referred to as a gene-specific second strand tailed primer. In some embodiments, the first and second gene-specific detection oligonucleotides bind to different sequences of the target gene that are about 50 to 600 nucleotides apart. Therefore, the first and second gene-specific detection oligonucleotides bind to different sequences of the target gene that are about 50, 60, 70, 80, 90, 100, 150, 200, 250, 300, 350, 400, 450, 500, 550, or 600 nucleotides apart.

In some embodiments, the methods include performing several steps of the library preparation within the partitions (FIGS. 3B-3E). In some embodiments, these steps include releasing the barcoding oligonucleotides from the beads. In some embodiments, these steps include performing a reverse transcription by annealing the first gene-specific detection oligonucleotide to cellular RNAs from the cell or nucleus and forming first strand cDNAs by extending the gene-specific detection oligonucleotides with the reverse transcriptase using the cellular RNAs as a template (top and middle panel of FIG. 3B). These steps produce RNA:DNA duplexes in the target gene, and the first strand cDNAs are tagged with the first tail capture sequence (middle panel of FIG. 3B). In some embodiments, the RNA in the RNA:DNA duplexes is degraded (middle panel of FIG. 3B). In some embodiments, second strand synthesis produces second strand cDNAs using first strand cDNAs as templates and the second gene-specific detection oligonucleotides as primers (bottom panel of FIG. 3B to top panel of FIG. 3C). The second strand cDNAs that are formed include 5′-3′: the second cleavable tail sequence, the target gene capture sequence, and a first DNA tail sequence that is a reverse complement sequence to the first cleavable tail sequence (top and middle panels of FIG. 3C). In some embodiments, the cleavable tail sequence is cleaved, creating a single-stranded first DNA tail sequence on the second strand cDNAs (middle panel of FIG. 3C). In some embodiments, the tail capture sequence of the first barcoding oligonucleotide is annealed to the single-stranded first DNA tail sequence on the second strand cDNAs, and strand synthesis is performed using a displacing polymerase and using the second strand cDNAs as templates and the first barcoding oligonucleotide to begin extension (bottom panel of FIG. 3C to middle panel of FIG. 3D). These steps produce double tailed first strand CDNAs that include 5′-3′: the first PCR priming sequence, the bead-specific barcode sequence, the first tail capture sequence, the portion of the target gene sequence, and the second DNA tail sequence that is a reverse complement sequence to the second cleavable tail sequence (middle panel of FIG. 3D). In some embodiments, strand synthesis is performed using the first barcoding oligonucleotide as a template and the first DNA tail sequence to begin extension (top panel of FIG. 3D). The second cleavable tail sequence is degraded, creating a single-stranded second DNA tail sequence on the double tailed first strand cDNAs (bottom panel of FIG. 3D). In some embodiments, the tail capture sequence of the second barcoding oligonucleotide anneals to the single-stranded second DNA tail sequence on the double tailed first strand cDNAs, and strand synthesis is performed using a displacing polymerase (bottom panel of FIG. 3D to middle panel of FIG. 3E). These steps produce double tailed second strand cDNAs using the double tailed first strand cDNAs as templates and the second barcoding oligonucleotide to begin extension (middle panel of FIG. 3E). The resulting double tailed second strand cDNAs include 5′-3′: the first PCR priming reverse complement sequence, the bead-specific barcode sequence, the second tail capture sequence, the target gene reverse complement sequence, the first DNA tail sequence, a bead-specific barcode reverse complement sequence, and the second PCR priming reverse complement sequence (bottom panel of FIG. 3E). In some embodiments, strand synthesis produces a double tailed first strand cDNA by using the second barcoding oligonucleotide as a template and the second DNA tail sequence to begin extension (middle and bottom panel of FIG. 3E).

In some embodiments, the methods include performing the remaining steps outside the individual partitions. These steps include generating a bulk mixture by combining contents of the partitions and performing an amplification (e.g., PCR) reaction with primers that bind to the first or second PCR priming sequences or first or second PCR priming reverse complement sequences. These steps produce double stranded sequencing cDNAs including 5′-3′: the first PCR priming sequence, the bead-specific barcode sequence, the first tail capture sequence, the target gene sequence, the second tail sequence, the bead-specific barcode reverse complement sequence, and the second PCR priming sequence. In some embodiments, the primers used in the amplification reaction include an index sequence, thereby forming double stranded sequencing cDNAs comprising the index sequence.

In some embodiments, the methods further include determining the nucleotide sequence of the double-stranded sequencing cDNAs and performing bead deconvolution. If two different bead-specific barcode sequences are present on the same cDNA, then sequencing reads comprising either of the two bead-specific barcode sequences are from the same partition.

The disclosure also provides pluralities of partitions (e.g., droplets in an emulsion or microwells) for use in this double tailed approach for barcoding cDNAs. In some embodiments, different partitions include at least one bead (e.g., a hydrogel bead) linked to a plurality of copies of a first barcoding oligonucleotide and a plurality of copies of a second barcoding oligonucleotide. In some embodiments, the first barcoding oligonucleotide includes 5′-3′: a first PCR priming sequence, a bead-specific barcode sequence, and a first tail capture sequence; and the second barcoding oligonucleotide includes 5′-3′: a second PCR priming sequence, the bead-specific barcode sequence, and a second tail capture sequence. In some embodiments, the partitions include a plurality of pairs of cleavable-tailed gene-specific detection oligonucleotides, wherein each pair includes a first gene-specific detection oligonucleotide that includes 5′-3′: a first cleavable tail sequence and a target gene sequence, and a second gene-specific detection oligonucleotide that includes 5′-3′: a second cleavable tail sequence and a target gene capture sequence. In some embodiments, the first and second gene-specific detection oligonucleotides bind to different sequences of the target gene that are about 50 to 600 nucleotides apart. In some embodiments, the partitions also include a reverse transcriptase, a DNA polymerase, and a single cell (e.g., a mammalian cell) or nucleus or nucleic acids from a single cell or nucleus.

D. Methods and Compositions of Barcoding cDNAs Using a Hybrid Tailed Approach

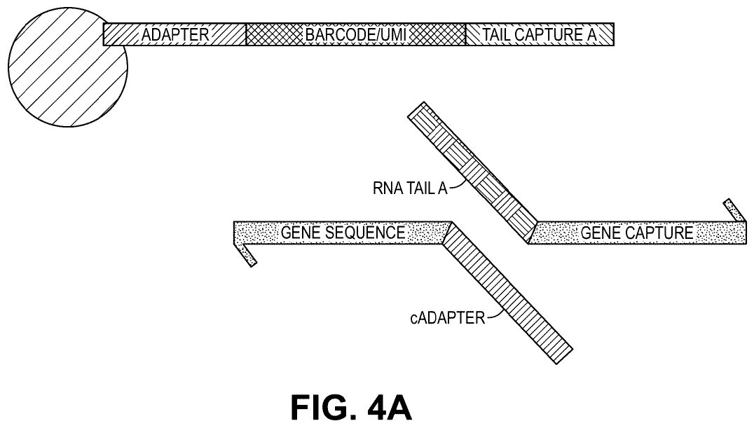

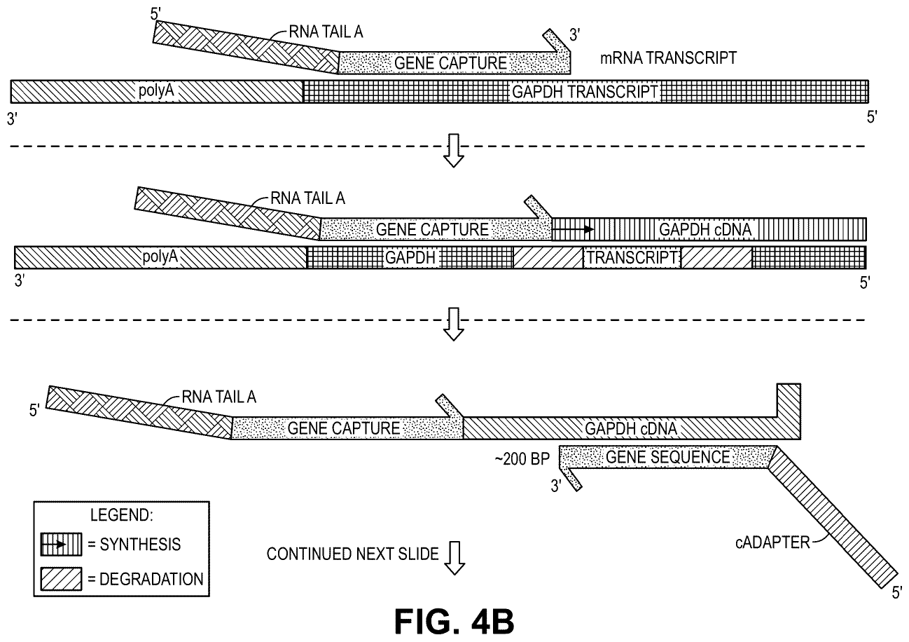

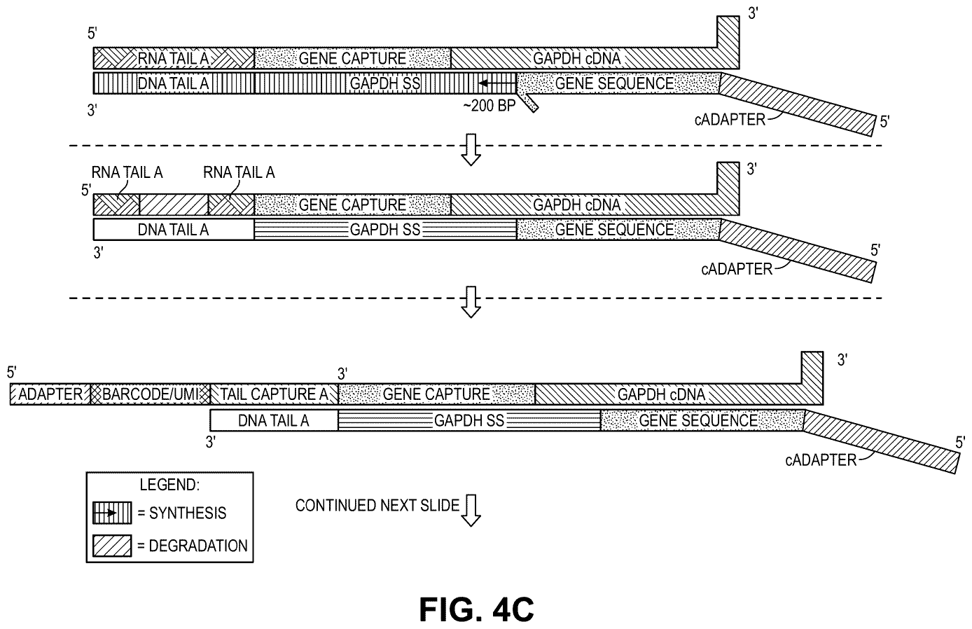

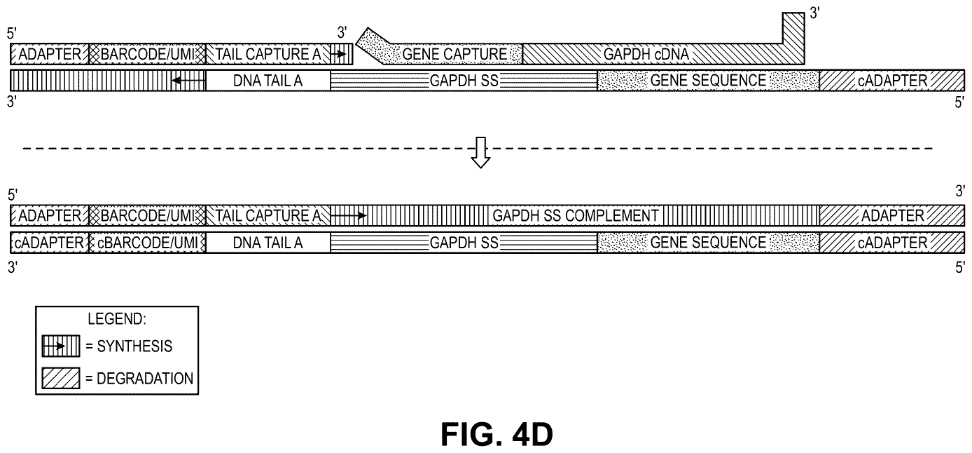

The disclosure also provides methods of barcoding cDNAs from cells in a partition using a hybrid tailed approach. In these embodiments, pairs of gene-specific detection oligonucleotides are used for each target gene. The first gene-specific detection oligonucleotide in each pair includes a tail sequence that is incorporated in the final product for sequencing. These methods produce a final product that is a double-stranded cDNA product for sequencing that includes a universal adapter sequence at both ends of the molecule. The final product also includes a bead-specific barcode sequence, a tail capture sequence, and a portion of the target gene that has a length suitable for sequencing (see, e.g., bottom panel in FIG. 4D). The description of various components and steps described for the methods in Sections A.-C. above (e.g., partitions, beads, cells, PCR priming sequences, universal adapter sequences, reverse transcription, RNA degradation, number and type of target genes, cleavable tail sequence, length of portion of target gene sequence present in the final product for sequencing) also applies to the methods described in this section unless otherwise noted in this section.

In some embodiments, the bead is linked to a plurality of copies of a barcoding oligonucleotide that has 5′-3′: a first PCR priming sequence, a bead-specific barcode sequence, and a tail capture sequence (FIG. 4A). In some embodiments, the pairs of gene-specific detection oligonucleotides include a first gene-specific detection oligonucleotide that includes 5′-3′: a cleavable tail sequence and a target gene sequence, and a second gene-specific detection oligonucleotide that includes 5′-3′: a second PCR priming reverse complement sequence and a target gene reverse complement sequence (FIG. 4A). The first gene-specific detection oligonucleotide also may be referred to as a gene-specific first strand tailed capture oligonucleotide (FSTC), and the second gene-specific detection oligonucleotide also may be referred to as a gene-specific second strand primer (SSP). In some embodiments, the first and second gene-specific detection oligonucleotides bind to different sequences of the target gene that are about 50 to 600 nucleotides apart. In some embodiments, the first and second gene-specific detection oligonucleotides bind to different sequences of the target gene that are about 50, 60, 70, 80, 90, 100, 150, 200, 250, 300, 350, 400, 450, 500, 550, or 600 nucleotides apart.

In some embodiments, the methods include performing several steps of the library preparation within the partitions. In some embodiments, these steps include releasing the barcoding oligonucleotides from the beads. In some embodiments, these steps include performing reverse transcription by annealing the first gene-specific detection oligonucleotide to cellular RNAs from the cell or nucleus and forming cleavable tailed first strand cDNAs by extending the first gene-specific detection oligonucleotides with reverse transcriptase using the cellular RNAs as a template, creating RNA:DNA duplexes (top and middle panels of FIG. 4B). The cleavable tailed first strand gene-specific cDNAs comprise the cleavable tail sequence and a portion of the target gene sequence. In some embodiments, the RNA is degraded (middle panel of FIG. 4B). In some embodiments, second strand synthesis produces second strand cDNAs using cleavable-tailed first strand cDNAs as templates and the second gene-specific detection oligonucleotides as primers (bottom panel of FIG. 4B to top panel of FIG. 4C). The second strand cDNAs include 5′-3′: the second PCR priming reverse complement sequence, the portion of the target gene reverse complement sequence, and a DNA tail sequence that is a reverse complement sequence to the cleavable tail sequence (top and middle panels of FIG. 4C). In some embodiments, the cleavable tail sequence is cleaved, creating a single-stranded DNA tail sequence on the second strand cDNAs (bottom panel of FIG. 4C). In some embodiments, the tail capture sequence of the barcoding oligonucleotide anneals to the single-stranded DNA tail sequence on the second strand cDNAs, and strand synthesis using a displacing polymerase produces tailed first strand cDNAs using the second strand cDNAs as templates and the barcoding oligonucleotide as a primer to begin extension (bottom panel of FIG. 4C to FIG. 4D). The tailed first strand cDNAs includes 5′-3′: the first PCR priming sequence, the bead-specific barcode sequence, the tail capture sequence, the portion of the target gene sequence, and the second PCR priming sequence. In some embodiments, strand synthesis using the barcoding oligonucleotide as a template and the DNA tail sequence as a primer to begin extension produces a tailed second strand cDNA (FIG. 4D).

In some embodiments, the methods include performing the remaining steps outside the individual partitions. These steps include generating a bulk mixture by combining contents of the partitions; and performing an amplification (e.g., PCR) reaction with primers that bind to the first or second PCR priming sequence or first or second PCR priming reverse complement sequence. These steps produce double stranded sequencing cDNAs that include 5′-3′: the first PCR priming sequence, the bead-specific barcode sequence, the tail capture sequence, the target gene sequence, and the second PCR priming sequence. In some embodiments, the primers used in the amplification reaction include an index sequence, thereby forming double stranded sequencing cDNAs comprising the index sequence.

The disclosure also provides pluralities of partitions (e.g., droplets in an emulsion or microwells) for use in this hybrid tailed approach for barcoding cDNAs. In some embodiments, the different partitions include at least one bead (e.g., hydrogel bead) linked to a plurality of copies of a barcoding oligonucleotide that includes 5′-3′: a first PCR priming sequence, a bead-specific barcode sequence, and a tail capture sequence. In some embodiments, the partitions include a plurality of pairs of gene-specific detection oligonucleotides. In certain embodiments, the first gene-specific detection oligonucleotide of each pair includes 5′-3′: a cleavable tail sequence and a target gene sequence, and the second gene-specific detection oligonucleotide of each pair includes 5′-3′: a second PCR priming reverse complement sequence and a target gene reverse complement sequence. In certain embodiments, the first and second gene-specific detection oligonucleotides bind to different sequences of the target gene that are about 50 to 600 nucleotides apart. In some embodiments, the partitions include a reverse transcriptase, a polymerase, and a single cell, single nucleus, or nucleic acids from a single cell or nucleus.

### Kits

In some embodiments, the disclosure provides kits for use in any of the disclosed methods for barcoding cDNAs from cells in a partition. In certain embodiments, the kits include at least one bead having a bead-specific barcoding oligonucleotide, at least one gene-specific detection oligonucleotide, a reverse transcriptase, and optionally a DNA polymerase, for example as described above or elsewhere herein.

In certain embodiments, the kits include at least one bead linked to a plurality of copies of a barcoding oligonucleotide comprising 5′-3′: a first PCR priming sequence, a bead-specific barcode sequence, and a capture sequence; a plurality of gene-specific detection oligonucleotides, wherein each gene-specific detection oligonucleotide comprises 5′-3′: a second PCR priming sequence and a portion of a target gene reverse complement sequence that is 50-600 nucleotides away from the capture sequence or DNA equivalent thereof; a reverse transcriptase; and optionally a polymerase.

In other embodiments, the kits include at least one bead linked to a plurality of copies of a first barcoding oligonucleotide and a plurality of copies of a second barcoding oligonucleotide, wherein the first barcoding oligonucleotide comprises 5′-3′: a first PCR priming sequence, a bead-specific barcode sequence, and a capture sequence, and a site labile to a single stranded break or nick; and wherein the second barcoding oligonucleotide comprises 5′-3′: a second PCR priming sequence, a bead-specific barcode sequence, and a tail capture sequence; a plurality of cleavable-tailed gene-specific detection oligonucleotides, wherein each gene-specific detection oligonucleotide comprises 5′-3′: a cleavable tail sequence and a portion of a target gene DNA reverse complement sequence; a reverse transcriptase; and optionally a DNA polymerase.

In further embodiments, the kits include at least one bead linked to a plurality of copies of a first barcoding oligonucleotide and a plurality of copies of a second barcoding oligonucleotide, wherein the first barcoding oligonucleotide comprises 5′-3′: a first PCR priming sequence, a bead-specific barcode sequence, and a first tail capture sequence; and wherein the second barcoding oligonucleotide comprises 5′-3′: a second PCR priming sequence, the bead-specific barcode sequence, and a second tail capture sequence; a plurality of pairs of cleavable-tailed gene-specific detection oligonucleotides, wherein each pair comprises a first gene-specific detection oligonucleotide that comprises 5′-3′: a first cleavable tail sequence and a target gene sequence and a second gene-specific detection oligonucleotide that comprises 5′-3′: a second cleavable tail sequence and a target gene capture sequence (e.g., target gene reverse complement sequence), wherein the first and second gene-specific detection oligonucleotides bind to different sequences of the target gene, and wherein the first gene-specific detection oligonucleotide binds to a sequence that is 50 to 600 nucleotides from the sequence to which the second gene-specific detection oligonucleotide binds; a reverse transcriptase; and optionally a DNA polymerase.

In other embodiments, the kits include at least one bead linked to a plurality of copies of a barcoding oligonucleotide, wherein the barcoding oligonucleotide comprises 5′-3′: a first PCR priming sequence, a bead-specific barcode sequence, and a tail capture sequence; a plurality of pairs of gene-specific detection oligonucleotides, wherein each pair comprises a first gene-specific detection oligonucleotide that comprises 5′-3′: a cleavable tail sequence and a target gene sequence and a second gene-specific detection oligonucleotide that comprises 5′-3′: a second PCR priming reverse complement sequence and a target gene reverse complement sequence, wherein the first and second gene-specific detection oligonucleotides bind to different sequences of the target gene, and wherein the first gene-specific detection oligonucleotide binds to a sequence that is 50 to 600 nucleotides from the sequence to which the second gene-specific detection oligonucleotide binds; a reverse transcriptase; and optionally a DNA polymerase.

### EXAMPLES

The following examples are offered to illustrate, but not to limit the claimed invention.

### Example 1—Materials and Methods

### Materials

Hydrogel beads with a universal adapter, barcodes, and poly T capture sequences were manufactured (Bio-Rad). Human cells (K-562) were obtained from ATCC. Enzymes used were Maxima RNase H Minus Reverse Transcriptase (Thermo), ddTaq (Bio-Rad), and USER (NEB). The ddSEQ platform and droplet generating reagents, including cell suspension buffer, bead suspension buffer, and cell encapsulation oil were acquired from Bio-Rad. Oligonucleotides, such as the gene-specific primer for GAPDH (including a universal adapter sequence and UMI) and the primers and probes for GAPDH-specific ddPCR, were obtained from IDT. Magnetic bead cleanups were performed using SPRIselect beads (Beckman Coulter). The ddPCR was performed on QX200 and QXONE instruments (Bio-Rad) using manufacturer protocols and reagents. Sequencing was performed on the NextSeq 2000 (Illumina) using manufacturer protocols and reagents.

### Methods

Cells were suspended in the cell suspension buffer, and beads were loaded in the bead suspension buffer with or without the Gene Specific Primer (GSP) for GAPDH. Cell and bead mixtures were loaded into the ddSEQ instrument for droplet generation using manufacturer protocols. Different conditions were tested for the binding of the GSP (facilitating the release of the cDNA from the RNA), including 5× heat denaturation (with Maxima RNase H Minuse Reverse Transcriptase for reverse transcription and ddTaq for GSP extension), 10× heat denaturation with the same conditions, or 5× heat denaturation and annealing after droplet disruption and SPRIselect cleanup. In cases where the GSP was added in-droplet, SPRIselect cleanup was performed after droplet disruption. After cleanup, 12 cycle indexing PCR using primers targeting the universal adapter from the bead sequence and the universal adapter from the GSP was performed to amplify library molecules that were successfully extended from the GSP. Next-Generation Sequencing was performed on some samples (10× heat denaturation condition), and the rest were assessed with GAPDH specific ddPCR.

### Example 2—cDNA Library Preparation

To generate a library molecule using the GSP with a universal adapter sequence, the cDNA must be released from the mRNA, and the GSP must bind the cDNA. A single GSP targeting GAPDH was used to test different methods of this library generation. Heat denaturation of the RNA:cDNA heteroduplex and sequential annealing cycles to allow for multiple attempts (10×) of GSP binding showed successful incorporation of the GSP sequencing adapter via NextSeq sequencing. Of the 8,302,200 sequencing reads, 6,936,902 of them passed filtering and showed a GAPDH sequence, indicating most of the molecules that went into the sequencing run after indexing PCR were GAPDH with both universal adapter sequences. Additionally, GAPDH specific ddPCR was used to quantify GAPDH copies after reverse transcription and compare them to GAPDH after indexing PCR to determine the efficiency of the indexing PCR, and therefore how efficiently the universal adapter sequence was incorporated onto the cDNA. The 5× heat denaturation and annealing of the GSP in droplet showed a 292-fold increase in GAPDH after 12 cycles of indexing PCR, indicating that some of the GAPDH (approximately 14% of the expected 2048-fold) incorporated the universal adapter from the GSP, but the majority of GAPDH did not. Samples that underwent SPRIselect cleanup prior to addition and 5× cycling of GSP, however, showed a 1,400-fold increase in GAPDH copies after 12 cycles of indexing PCR, indicating that more than two-thirds of the GAPDH molecules (2,048 expected fold increase) have the universal adapter sequence from the successful binding of the GSP. This is significantly higher than previously collected data using a workflow with random priming universal adapter addition and fragmentation, which had a 280-fold increase after the 12 cycles (approximately 14% efficient).

### Example 3—cDNA Library Preparation for IgM Transcript

The cDNA library preparation methods were assessed using IgM as the gene of interest.

### Materials and Methods

Human Raji cells were acquired from the American Type Culture Collection (ATCC). Hydrogel beads with a universal adapter, barcodes, and gene specific capture sequences were manufactured (Bio-Rad). Enzymes used were Maxima RNase H Minus Reverse Transcriptase (Thermo), Go taq (Promega), and USER (NEB). The ddSEQ platform and droplet generating reagents, including cell suspension buffer, bead suspension buffer, and cell encapsulation oil were acquired from Bio-Rad Laboratories. Oligonucleotides, such as the gene-specific primer for B cell receptor (BCR) genes and the primers and probes for BCR specific ddPCR, were obtained from IDT. Magnetic bead cleanups were performed using SPRIselect beads (Beckman Coulter). Gel electrophoresis was performed on TapeStation instrument (Agilent) using manufacturer protocols and reagents. The ddPCR was performed on QX200 and QXONE instruments (Bio-Rad) using manufacturer protocols and reagents.

Cells were suspended in the cell suspension buffer, and beads were loaded in the bead suspension buffer. Cell and bead mixtures were loaded into the ddSEQ instrument for droplet generation using manufacturer protocols. Lysis and reverse transcription were done in droplets. After droplet disruption and SPRIselect cleanup, reverse transcription efficiency was assessed for some samples using gene-specific ddPCR. For other samples, after cleanup, targeted second strand synthesis using a gene-specific primer (GSP) followed by 20 cycle linear or exponential enrichment was performed. In cases where 20 cycle linear enrichment was performed, the GSP was used as the sole primer for enrichment. Then, SPRIselect cleanup was performed followed by 20 cycle exponential enrichment using primers targeting the universal adapter from the bead sequence and the same GSP used in linear enrichment to amplify the library. Workflow efficiency was assessed by gene specific ddPCR as described below.

ddPCR Assays

For assessing reverse transcription efficiency, two ddPCR assays were used. A first assay targeting the capture site on the gene (3′ assay), and a second assay targeting the 5′ end of the gene (5′ assay). The purpose of using these assays was to assess the molecules that not only had the IgM constant sequence, but also included the 5′ end of the molecule (i.e., a full length transcript). The ratio of positive droplets in the assays indicate whether the captured molecules are truncated or full length.

### Linear Enrichment vs Exponential Enrichment

After lysis and reverse transcription in droplets, droplets were disrupted and SPRIselect cleanup was performed. Two approaches were tested. Approach 1 used targeted second strand synthesis followed by 20 cycles of linear enrichment using a GSP targeting the 5′ end of the gene. After enrichment, SPRIselect cleanup was done followed by 20 cycle exponential enrichment using the same GSP targeting the 5′ end of the gene and a primer targeting the universal adapter from the bead sequence. Finally, a SPRIselect cleanup was done prior to ddPCR. By contrast, Approach 2 used 20 cycles of exponential enrichment using GSP targeting the 5′ end of the gene and a primer targeting the universal adapter from the bead sequence. SPRIselect cleanup was done prior to ddPCR.

### Sequencing

Libraries were tagmented and indexed using Nextera enzymes and indexes from Illumina, then sequenced on a Nextseq 2000 sequencer from Illumina using a P1 paired read kit (2×150 bp). Assemblies were generated from tagmented reads with Trinity.

### Results

After running a single cell reverse transcription reaction in droplet partitions, the transcripts of a gene of interest (IgM) were selectively enriched and made into sequencing libraries using either the disclosed approach (targeted second strand synthesis followed by a linear enrichment) followed by an exponential PCR (Approach 1) or directly into an exponential PCR only approach (Approach 2). The target transcripts (expected size approximately 580 bp) from Approach 2 were undetectable on the High Sensitivity D1000 screening tape (FIG. 1D), while a clear visible peak was seen in Approach 1, with the earlier enrichment step (FIG. 1E). Performing ddPCR on those samples to quantify the IgM copies per cell from both ends of the transcript (a separate 5′ assay and 3′ assay) showed that only 12% of the copies of IgM were full length after PCR for Approach 2, compared to 94% in Approach 1 after the exponential PCR, with the number of full length copies being almost 1,000 fold higher at 50 billion full length IgM using Approach 1, as compared to 67 million in Approach 2. This indicated that performing the targeted second strand synthesis and linear enrichment significantly improved the recovery of the full-length IgM transcripts and produced a better-quality sequencing library compared to using PCR for enrichment and adapter addition.

To test how effective each approach would be in assembling the target region of the transcripts (approximately 580 bp) using paired end sequencing (2×150 bp), tagmentation was performed on the libraries, and they were sequenced and reassembled using Trinity software. Out of the approximately 250 input cells, productive IgM assemblies were recovered from less than 10 of them in the PCR only Approach 2 (FIG. 1F, left panel), while approximately 100 were recovered using the disclosed Approach 1. This trend was also prevalent when analyzing for complete CDR3 regions, which are of immunological significance (FIG. 1F, right panel). This demonstrates that the targeted second strand synthesis and linear enrichment in Approach 1 outperformed Approach 2 (exponential PCR only) in both ddPCR quantification of a target full length transcript as well as in next generation sequencing.

Although the foregoing disclosure has been described in some detail by way of illustration and example for purposes of clarity of understanding, one of skill in the art will appreciate that certain changes and modifications may be practiced within the scope of the appended claims. In addition, each reference provided herein, including patents, patent applications, non-patent literature, and GenBank accession numbers, is incorporated by reference in its entirety to the same extent as if each reference was individually incorporated by reference. Where a conflict exists between the instant application and a reference provided herein, the instant application shall dominate.

1. A method of barcoding cDNAs from cells in a partition, the method comprising:
providing a plurality of partitions, wherein different partitions comprise:
(i) at least one bead linked to a plurality of copies of barcoding oligonucleotides comprising 5′-3′: a first PCR priming sequence, a bead-specific barcode sequence, and a capture sequence; and
(ii) a single cell, single nucleus, or nucleic acids from a single cell or nucleus;
in the partitions, performing RNA capture, wherein the performing comprises annealing sequences of cellular RNA to some copies of the barcoding oligonucleotides;
performing reverse transcription, wherein performing comprises forming first strand cDNAs by extending the barcoding oligonucleotides with reverse transcriptase using the cellular RNA as a template, thereby creating RNA:DNA duplexes, wherein the first strand cDNAs comprise the first PCR priming sequence, the bead-specific barcode sequence, the capture sequence, and a portion of the target gene sequence;
removing at least a portion of the RNA in the RNA:DNA duplexes;
contacting the first strand cDNAs with a plurality of gene-specific detection oligonucleotides, wherein each gene-specific detection oligonucleotide comprises 5′-3′: a second PCR priming sequence and a portion of a target gene cDNA reverse complement sequence, wherein the portion is 50-600 nucleotides away from the capture sequence or the DNA equivalent thereof;
performing second strand synthesis to form second strand cDNAs using first strand cDNAs as templates and the gene-specific detection oligonucleotides as primers, thereby forming double-stranded cDNAs of sequencing length from a plurality of different RNAs; and
performing a PCR reaction using a DNA polymerase with the double-stranded cDNAs, a first primer comprising a universal adapter sequence and a bead-specific barcode sequence or reverse complement sequence, and a second primer that comprises a universal adapter sequence and a target gene sequence or reverse complement sequence; thereby producing double stranded sequencing cDNA libraries comprising 5′-3′; the universal adapter sequence, the bead-specific barcode sequence, and the portion of the target gene sequence.

2. The method of claim 1, wherein the first primer, the second primer, or both comprise an index sequence, thereby forming double stranded sequencing cDNAs comprising the index sequence.

3. The method of claim 1, wherein the capture sequence comprises 22-36 Ts.

4. The method of claim 1, wherein reverse transcription or both reverse transcription and second strand synthesis are performed in the partition.

5. (canceled)

6. The method of claim 1, wherein the plurality of gene-specific detection oligonucleotides comprises at least 100 gene-specific detection oligonucleotides specific for different mRNAs present in the single cell or nucleus.

7. The method of claim 1, wherein at least one gene-specific detection oligonucleotide of the plurality of gene-specific detection oligonucleotides is specific for a target gene that is known to be expressed in the single cell.

8. The method of claim 1, wherein at least one gene-specific detection oligonucleotide binds to a sequence that is 100 to 400 nucleotides upstream of the mRNA poly A tail.

9. The method of claim 8, wherein at least one gene-specific detection oligonucleotide binds to a sequence that is 180 to 220 nucleotides upstream of the mRNA poly A tail.

10. The method of claim 1, wherein the providing a plurality of partitions comprises providing partitions comprising intact cells and subsequently lysing the cells in the partitions.

11. The method of claim 1, wherein the reverse transcription and the RNA:DNA duplex degradation are performed by a single enzyme having RNase H⁺ activity.

12. The method of claim 1, further comprising, after performing second strand synthesis:
inactivating the polymerase and reverse transcriptase in the partitions.

13. The method of claim 12, wherein the inactivating comprises applying heat to the partitions.

14. The method of claim 13, wherein the inactivating comprises incubating the partitions at 75-90 degrees Celsius.

15. The method of claim 1, wherein the partitions are droplets in an emulsion or microwells.

16. The method of claim 1, wherein the cell is a mammalian cell.

17. The method of claim 1, wherein the bead is a hydrogel bead.

18. A plurality of partitions, wherein different partitions comprise:
(i) at least one bead linked to a plurality of copies of a barcoding oligonucleotide comprising 5′-3′: a shared priming sequence, a bead-specific barcode sequence, and a capture sequence;
(ii) a plurality of gene-specific detection oligonucleotides, wherein each gene-specific detection oligonucleotide comprises 5′-3′: a shared priming reverse complement sequence and a portion of a target gene reverse complement sequence wherein the portion is 50-600 nucleotides away from the capture sequence or the DNA equivalent thereof;
(iii) a reverse transcriptase;
(iv) a polymerase; and
(v) a single cell, single nucleus, or nucleic acids from a single cell or nucleus.

19. The plurality of partitions of claim 18, wherein the partitions are droplets in an emulsion or microwells.

20. The plurality of partitions of claim 18, wherein the cell is a mammalian cell.

21. The plurality of partitions of claim 18, wherein the bead is a hydrogel bead.

22. A method of barcoding cDNAs from cells in a partition, the method comprising:
(A)
providing a plurality of partitions, wherein different partitions comprise:
(i) at least one bead linked to a plurality of copies of a first barcoding oligonucleotide and a plurality of copies of a second barcoding oligonucleotide; wherein the first barcoding oligonucleotide comprises: a first PCR priming sequence, a bead-specific barcode sequence, a capture sequence, and a site labile to a single stranded break or nick; and wherein the second barcoding oligonucleotide comprises 5′-3′: a second PCR priming sequence, a bead-specific barcode sequence, and a tail capture sequence;
(ii) a plurality of cleavable-tailed gene-specific detection oligonucleotides, wherein each gene-specific detection oligonucleotide comprises: a cleavable tail sequence and a portion of a target gene DNA reverse complement sequence that binds to the cDNA 50-600 bp away from the capture sequence;
(iii) a reverse transcriptase; and
(iv) a single cell, nucleus, or nucleic acids from a single cell or nucleus;
in the partitions, performing reverse transcription, wherein the performing comprises annealing sequences of the cellular RNAs to some copies of the first barcoding oligonucleotides and forming first strand cDNAs by extending the first barcoding oligonucleotides with the reverse transcriptase using the cellular RNAs as a template, thereby creating RNA:DNA duplexes, wherein the first strand cDNAs comprise the first PCR priming sequence, the bead-specific barcode sequence, the capture sequence, the site labile to a single stranded break or nick, and the target gene sequence;
in the partitions, degrading the RNA in the RNA:DNA duplexes;
in the partitions, performing a second strand synthesis to form second strand gene-specific cDNAs including the cleavable tail using first strand cDNAs as templates and the gene-specific detection oligonucleotides as primers, thereby forming double-stranded cDNAs wherein the tailed second strand gene-specific cDNAs comprise: the cleavable tail sequence, the target gene reverse complement sequence, the bead-specific barcode reverse complement sequence, and the first PCR priming reverse complement sequence;
in the partitions, producing a single stranded break at the labile site, thereby creating a free 3′ hydroxyl group at the site;
in the partitions, performing a strand synthesis with a displacing polymerase to form DNA-tailed first strand cDNAs using the second strand gene-specific cDNAs as templates and the free 3′ hydroxyl group to begin extension, thereby forming tailed double-stranded cDNAs, wherein the DNA-tailed first strand cDNAs comprise: the first PCR priming sequence, the bead-specific barcode sequence, the target gene sequence, and a DNA tail sequence that is reverse complementary to the cleavable tail sequence;
in the partitions, cleaving the cleavable tail sequence in the double stranded cDNAs, thereby producing a double-stranded cDNA with a single-stranded DNA tail on the 3′ end of the first strand;
in the partitions, annealing the DNA tail of the double-stranded cDNA to the second barcoding oligonucleotide and performing a strand synthesis using the second barcoding oligonucleotide as templates and the single-stranded DNA tail of the first strand cDNA to begin extension;
in the partitions, performing a strand synthesis with a displacing polymerase to form double-stranded cDNAs comprising: the first PCR priming sequence, the bead-specific barcode sequence, the truncated capture sequence, the target gene sequence, the DNA tail sequence, the bead-specific barcode reverse complement sequence, and the second PCR priming reverse complement sequence;
generating a bulk mixture by combining contents of the partitions; and
performing an amplification reaction with primers that bind to the first or second PCR priming sequence or reverse complement sequence, thereby forming double stranded sequencing cDNA libraries comprising: the PCR priming sequence, the bead-specific barcode sequence, the capture sequence, the target gene sequence, the DNA tail sequence, the bead-specific barcode reverse complement sequence, and the PCR priming reverse complement sequence;
or
(B)
providing a plurality of partitions, wherein different partitions comprise:
(i) at least one bead linked to a plurality of copies of a first barcoding oligonucleotide and a plurality of copies of a second barcoding oligonucleotide, wherein the first barcoding oligonucleotide comprises 5′-3′: a first PCR priming sequence, a bead-specific barcode sequence, and a first tail capture sequence; and wherein the second barcoding oligonucleotide comprises 5′-3′: a second PCR priming sequence, the bead-specific barcode sequence, and a second tail capture sequence;
(ii) a plurality of pairs of cleavable-tailed gene-specific detection oligonucleotides, wherein each pair comprises a first gene-specific detection oligonucleotide that comprises a first cleavable tail sequence and a target gene sequence and a second gene-specific detection oligonucleotide that comprises a second cleavable tail sequence and a target gene capture sequence, wherein the first and second gene-specific detection oligonucleotides bind to different sequences of the target gene, and wherein the first gene-specific detection oligonucleotide binds to a sequence that is 50 to 600 nucleotides from the sequence to which the second gene-specific detection oligonucleotide binds;
(iii) a reverse transcriptase; and
(iv) a single cell, nucleus, or nucleic acids from a single cell or nucleus;
in the partitions, performing a reverse transcription, wherein the performing comprises annealing the first gene-specific detection oligonucleotide to cellular RNAs from the cell or nucleus and forming first strand cDNAs by extending the gene-specific detection oligonucleotides with the reverse transcriptase using the cellular RNAs as a template, thereby creating RNA:DNA duplexes, wherein the first strand cDNAs comprise the first cleavable tail sequence and the target gene sequence;
in the partitions, degrading the RNA in the RNA:DNA duplexes;
in the partitions, performing a second strand synthesis to form second strand cDNAs using first strand cDNAs as templates and the second gene-specific detection oligonucleotides as primers, thereby forming double-stranded cDNAs, wherein the second strand cDNAs comprise the second cleavable tail sequence, the target gene capture sequence, and a first DNA tail sequence that is a reverse complement sequence to the first cleavable tail sequence;
in the partitions, cleaving the first cleavable tail sequence, thereby creating a single-stranded first DNA tail sequence on the second strand cDNAs;
in the partitions, annealing the tail capture sequence of the first barcoding oligonucleotide to the single-stranded first DNA tail sequence on the second strand cDNAs and performing strand synthesis using a displacing polymerase to form double tailed first strand cDNAs using the second strand cDNAs as templates and the first barcoding oligonucleotide to begin extension, wherein the double tailed first strand cDNAs comprise the first PCR priming sequence, the bead-specific barcode sequence, the first tail capture sequence, the target gene sequence, and the second DNA tail sequence that is a reverse complement sequence to the second cleavable tail sequence; and performing strand synthesis using the first barcoding oligonucleotide as a template and the first DNA tail sequence to begin extension;
in the partitions, degrading the second cleavable tail sequence, thereby creating a single-stranded second DNA tail sequence;
in the partitions, annealing the tail capture sequence of the second barcoding oligonucleotide to the single-stranded second DNA tail sequence and performing strand synthesis using a displacing polymerase and the double tailed first strand cDNAs as templates and the second barcoding oligonucleotide to begin extension, thereby forming double tailed second strand cDNAs comprising the PCR priming reverse complement sequence, the bead-specific barcode sequence, the second tail capture sequence, the target gene reverse complement sequence, the first DNA tail sequence, a bead-specific barcode reverse complement sequence, and the second PCR priming reverse complement sequence; and performing strand synthesis to form a double tailed first strand cDNA using the second barcoding oligonucleotide as a template and the second DNA tail sequence to begin extension;
generating a bulk mixture by combining contents of the partitions; and performing a PCR reaction with primers that bind to the PCR priming sequences or reverse complement sequences, thereby forming double stranded sequencing cDNA libraries, wherein the cDNAs comprise the first PCR priming sequence, the bead-specific barcode sequence, the first tail capture sequence, the target gene sequence, the second tail sequence, the bead-specific barcode reverse complement sequence, and the second PCR priming sequence;
or
(C)
providing a plurality of partitions, wherein different partitions comprise:
(i) at least one bead linked to a plurality of copies of a barcoding oligonucleotide, wherein the barcoding oligonucleotide comprises 5′-3′: a PCR priming sequence, a bead-specific barcode sequence, and a tail capture sequence;
(ii) a plurality of pairs of gene-specific detection oligonucleotides, wherein each pair comprises a first gene-specific detection oligonucleotide that comprises a cleavable tail sequence and a target gene sequence, and a second gene-specific detection oligonucleotide that comprises a second PCR priming reverse complement sequence and a target gene reverse complement sequence, wherein the first and second gene-specific detection oligonucleotides bind to different sequences of the target gene, and wherein the first gene-specific detection oligonucleotide binds to a sequence that is 50 to 600 nucleotides from the sequence to which the second gene-specific detection oligonucleotide binds;
(iii) a reverse transcriptase; and
(iv) a single cell, single nucleus, or nucleic acids from a single cell or nucleus;
in the partitions, performing a reverse transcription, wherein the performing comprises annealing the first gene-specific detection oligonucleotide to cellular RNAs from the cell or nucleus and forming cleavable tailed first strand gene-specific cDNAs by extending the first gene-specific detection oligonucleotides with reverse transcriptase using the cellular RNAs as a template, thereby creating RNA:DNA duplexes, wherein the cleavable tailed first strand gene-specific cDNAs comprise the cleavable tail sequence and the target gene sequence;
in the partitions, degrading the RNA in the RNA:DNA duplexes;
in the partitions, performing second strand synthesis to form second strand cDNAs using cleavable tailed first strand cDNAs as templates and the second gene-specific detection oligonucleotides as primers, thereby forming double-stranded cDNAs, wherein the second strand cDNAs comprise the second PCR priming reverse complement sequence, the target gene reverse complement sequence, and a DNA tail sequence that is a reverse complement sequence to the cleavable tail sequence;
in the partitions, cleaving the cleavable tail, thereby creating a single-stranded DNA tail sequence on the second strand cDNAs;
in the partitions, annealing the tail capture sequence of the barcoding oligonucleotide to the single-stranded DNA tail sequence on the second strand cDNAs and performing strand synthesis using a displacing polymerase to form tailed first strand cDNAs using the second strand cDNAs as templates and the barcoding oligonucleotide as a primer to begin extension, wherein the tailed first strand cDNAs comprise the first PCR priming sequence, the bead-specific barcode sequence, the tail capture sequence, the target gene sequence, and the second PCR priming sequence; and performing strand synthesis to form a tailed second strand cDNA using the barcoding oligonucleotide as a template and the DNA tail sequence as a primer to begin extension;
generating a bulk mixture by combining contents of the partitions; and performing a PCR reaction with primers that bind to the first or second PCR priming sequences or reverse complement sequences, thereby forming double stranded sequencing cDNA libraries, wherein the cDNAs comprise the first PCR priming sequence, the bead-specific barcode sequence, the tail capture sequence, the target gene sequence, and the second PCR priming sequence.

23.-89. (canceled)


SEQUENCE LISTING (7 sequences)

SEQ ID NO: 1-2 - skipped (2 entries)

SEQ ID NO: 3 - DNA, 17 bp, synthetic construct
  source 1..17: mol_type = other DNA
aatgatactgcgaccga

SEQ ID NO: 4 - DNA, 24 bp, synthetic construct
  source 1..24: mol_type = other DNA
caagcagaagacggcatacgagat

SEQ ID NO: 5 - DNA, 41 bp, synthetic construct
  source 1..41: mol_type = other DNA
ccactacgcctccgctttcctctctatgggcagtcggtgat

SEQ ID NO: 6 - DNA, 30 bp, synthetic construct
  source 1..30: mol_type = other DNA
ccatctcatccctgcgtgtctccgactcag

SEQ ID NO: 7 - DNA, 36 bp, unidentified
  source 1..36: mol_type = unassigned DNA
  misc_difference 1..36: note = This sequence may encompass 22-36 nucleotides
tttttttttttttttttttttttttttttttttttt